(19) United States
(12) Patent Application Publication
(10) Pub. No.: US 2021/0166578 A1
(43) Pub. Date: Jun. 3, 2021

(54) VISUAL APPROACH-BASED APTITUDE TESTING SYSTEM

(71) Applicant: JINSOKEN CO., LTD., Tokyo (JP)

(72) Inventor: Koichi NISHIOKA, Tokyo (JP)

(73) Assignee: JINSOKEN CO., LTD., Tokyo (JP)

(21) Appl. No.: 17/132,688

(22) Filed: Dec. 23, 2020

Related U.S. Application Data
(63) Continuation of application No. PCT/JP2019/024720, filed on Jun. 21, 2019.

(30) Foreign Application Priority Data
Jun. 27, 2018 (JP) 2018-121572

(51) Int. Cl. G09B 19/00

(52) U.S. Cl. CPC G09B 19/00

(57) ABSTRACT

A visual approach-based aptitude testing system includes a computer connected to an examinee's terminal through a communication network. The computer provides, to the examinee's terminal, a check sheet in which a plurality of areas are formed by boundary elements depicted in a frameless test area and which includes graphic figures each having no inherent meaning, and a plurality of icons applicable to the check sheet. The computer sequentially stores test data including icons applied by the examinee to the check sheet and an examinee's ID transmitted from the terminal, and sequentially reads out the test data and the examinee's ID to count a total number of the icons. The computer executes discrimination of whether the arrangement of the icons in the read-out test data falls under definitions in tables stored by the computer. Results of the discrimination are stored in the computer and used for evaluation of the examinee's aptitude.

### CROSS-REFERENCE TO RELATED APPLICATIONS

This application is a Continuation of International Application No. PCT/JP2019/024720, filed Jun. 21, 2019, and claims the benefit of priority of Japanese patent Application No. 2018-121572, filed Jun. 27, 2018, the entire contents of both of which are incorporated herein by reference.

### TECHNICAL FIELD

The present invention relates to a system for multifacetedly grasping and judging human psychiatric disciplines through visual information, and more particularly, to a visual approach-based aptitude testing system for judging how well an examinee is qualified for a particular occupation.

### BACKGROUND ART

Human behavior is a cross-over of various impacts derived from psychological situations and has a relationship with basic potential and performance potential. Various external stimuli may act as a burden when engaging in business, and psychosomatic stress may occur in such cases. Since stress is invisible in most cases, it is often difficult to visually and accurately grasp business-related stress.

In addition, concerning how to evaluate human performance in business, concentration (ability to work) and wage differentials derivable therefrom (rewarding money) have been considered from the viewpoint of market value.

However, since conventional performance theory is a rigid structural system in which the analytical result is difficult to reuse, it is difficult to make an appropriate determination from the results of an aptitude test based on the theory.

In addition, there is diversity in people's social behavior. The frontal lobes have been said to suppress the selfish “old brain,” as an explanation for why diverse people cooperate with each other.

Recently, however, the theory that the emotional system avoids inequity has become dominant. More specifically, the predictive accuracy of purchasing behavior, IQ estimates from resting fMRI, and MRI-based predictions indicate that people have a mechanism to avoid unfairness, and that the amygdala, which is a deep part of the “old brain,” reacts intuitively and automatically to avoid unfairness and promote prosocial behavior.

Prosocial behavior refers to behavior that maximizes harmony between oneself and others and minimizes differences therebetween, which will differ from individualistic behavior that maximizes oneself and also from competitive behavior that maximizes differences between oneself and others.

Analysis of fMRI data shows that the absolute value of reward difference and the activity of the amygdala are correlated with each other in prosocial behavior, and prosocial activity of the amygdala becomes active when there is a large absolute value of the reward difference.

The results of the ultimatum game experiment and the trust game experiment support the above theory, and there is a possibility that social disparity and depressive tendencies are related with each other.

As a result of fMRI analysis, it has been proved that the prefrontal area (frontal association area) or neocortex, which is evolutionarily new and constitutes the center of high order cognitive function, expresses guilt, and that the evolutionarily primitive amygdala and nucleus accumbens express inequality.

In addition, ⅓ of the cerebral cortex is related to the visual sense.

Traditionally, it has been said that competition creates progress (Eiichi Shibuya); only comparing yourself with others is social slavery (Rousseau); and comparing with others is the shortest path to become unfortunate (Buddha).

However, recent dominant theories have reported that social disparities are correlated with depressive tendencies.

Summarizing the above, recent advances in brain function imaging technologies have revealed that some kind of human social decision-making in the brain is based primarily in the subcortical area and cerebral cortex.

In addition, the frontal lobe (neocortex) relates to conscious information processing, the amygdala relates to emotional information processing such as fear and pleasure, and the septum and hippocampus relate to memory and emotion control. Considering the structures of the brain, people have two minds, one is the conscious, determinable mind, mainly existing in the frontal lobe, and the other is unconscious emotion, mainly existing in amygdala.

For some subconscious and apparent awareness, perceptual and behavioral determinations, including human subjective consciousness, are known to be made in the frontal lobe (neocortex). On the other hand, emotion (response of the amygdala) is not directly conscious.

The degree of development of the frontal lobe (neocortex) differs between adults and children. In other words, the response by the frontal lobe (neocortex) is considered to be low for children. Actually, a human perceives the result of expression of emotion to the human body, and recognizes one's emotion by comparing it with previous experiences, and expresses the same as emotions. In addition, it is said that some behaviors are not conscious as in custom, but are reflexive.

The present invention has been made in view of study and analysis of vast literature in the following areas and results from further creative improvements by the inventor:

<1> Evolutionary approaches to psychological demands and decision making,

<2> Stress observation,

<3> Edge effects,

<4> The moiré phenomenon,

<5> Human performance,

<6> Stress Theory,

<7> Psychological and physiological approaches to emotions,

<8> General adaptation syndrome (GAS) Theory,

<9> Information from brain science, cognitive science and neuroscience.

### SUMMARY OF INVENTION

In view of the above-mentioned background, an object of the present invention is to grasp human mental structure in a multifaceted manner through visual information, and to accurately and objectively grasp an examinee's propensity such as a vocational aptitude.

To achieve the above object, a visual approach-based aptitude testing system according to the present invention is an aptitude testing system including a computer connected to an examinee's terminal through a communication network.

The computer includes a memory which stores (i) information for creating a display screen including a check sheet in which a plurality of areas are formed by boundary elements depicted in a frameless test area and which includes graphic figures having no inherent meaning, and a plurality of icons applicable to the check sheet, and (ii) a first table including definitions of the boundary elements in the check sheet, a second table including kinds, a number, and definitions of the icons, and a third table including definitions of meanings of positions of the icons in the check sheet. The computer also includes a processor which executes a program to perform operations including: providing the information for creating the display screen to the examinee's terminal, so that the examinee's terminal displays the display screen including the check sheet and the plurality of icons; sequentially writing, to the memory, test data including icons applied by the examinee to the check sheet via the terminal, and an examinee's ID transmitted from the terminal; sequentially reading out the test data and the examinee's ID stored in the memory to count a total number of the icons applied to the check sheet; executing discrimination of whether or not an arrangement of the icons on the check sheet in the read-out test data falls under the definitions in the tables stored in the memory; and storing results of the discrimination in the memory to be used for evaluation of the examinee's aptitude based on the total number of the icons applied to the check sheet and the arrangement of the icons.

According to a second aspect of the present invention, the boundary elements include lines and/or corners.

According to a third aspect of the present invention, the lines include straight lines, curved lines, broken lines, or any combinations thereof.

According to a fourth aspect of the present invention, the corners include corners of a virtual frame, and/or points of intersection between lines.

According to a fifth aspect of the present invention, the memory further stores a fourth table which predefines a meaning of a positional relationship (hereinafter also referred to as an “icon relationship”) between a plurality of the icons or between one of the icons and one of the lines, and the operations performed by the processor further include executing discrimination of whether or not the icon relationship exists in the arrangement of the icons in the read-out test data, with reference to the fourth table.

According to a sixth aspect of the present invention,—the memory further stores a fifth table which predefines the meanings of the applied icons forming a specific pattern, and the operations performed by the processor further include executing discrimination of whether or not the predefined specific pattern exists.

According to a seventh aspect of the present invention, the memory further stores a sixth table which predefines a meaning of an appearance of a specific one of the icons, and the operations performed by the processor further include executing discrimination of whether or not the specific icon appears.

According to an eighth aspect of the present invention, the specific pattern includes at least one of a crowded arrangement, a dispersive arrangement, a balanced arrangement, a deviative arrangement, a bottom arrangement, an upper arrangement, a top-and-bottom arrangement, a left-and-right arrangement, a protruding arrangement, and a doll arrangement.

According to a ninth aspect of the present invention, the icon relationship includes at least one of an overlapping, contact, line-contact, and close-vicinity relationship.

According to a tenth aspect of the present invention, the boundary elements, which are depicted in the frameless test area and form the plurality of areas, comprise (i) a circular figure having a cut-out at a part of its circumference, (ii) a triangular figure having a cut-out at its apex and an intermediate line extending between opposite oblique side lines of the triangular figure and in parallel with a bottom of the triangular figure, (iii) a straight line extending obliquely upward and rightward from one end of the intermediate line to a window virtual line, (iv) a broken line extending obliquely upward and leftward from one end of the bottom of the triangular figure to a window virtual line, and (v) an S-shaped curved line extending downward from the bottom of the triangular figure to a window virtual line.

According to an eleventh aspect of the present invention, the boundary elements, which are depicted in the frameless test area and form the plurality of areas, comprise (i) a circular figure having a cut-out at a part of its circumference, (ii) a triangular figure having a cut-out at its apex and an intermediate line extending between opposite oblique side lines of the triangular figure and in parallel with a bottom of the triangular figure, (iii) a straight line extending obliquely downward from one end of the intermediate line to a window virtual line, (iv) a broken line extending obliquely upward from an intermediate location on the straight line to a lower portion of the circular figure, and (v) an S-shaped curved line extending upward from the bottom of the triangular figure to a window virtual line.

According to a twelfth aspect of the present invention, the boundary elements, which are depicted in the frameless test area and form the plurality of areas, comprise (i) a circular figure having a cut-out at a part of its circumference, (ii) a triangular figure having a cut-out at its apex and an intermediate line extending between opposite oblique side lines of the triangular figure and in parallel with a bottom of the triangular figure, (iii) a first straight line extending obliquely upward from a first end of the intermediate line to a window virtual line, (iv) a broken line extending obliquely upward and leftward from a first end of the bottom of the triangular figure to a window virtual line, (v) an S-shaped curved line extending downward from an intermediate portion of the bottom of the triangular figure to a window virtual line, (vi) a second straight line extending from an end of the cut-out of the circular figure to an end of the cutout of the triangular figure, and (vii) a third straight line which is inclined and extends from a second end of the intermediate line to a second end of the bottom of the triangular figure.

According to the visual approach-based aptitude testing system of the present invention, it is possible to visually and specifically express the examinee's internal and potential consciousness through the images obtained from the application state of icons on a check sheet, more particularly the number and position of the icons applied by the examinee. Thus, the examinee's mental structure can be grasped in a multifaceted manner through vision.

As mentioned above, one-third of the cerebral cortex is involved in vision. To visual stimuli i.e. visual information, the amygdala, which is the emotional brain (i.e., controls emotion) will respond faster than the neocortex which is said to conduct cognitive processing. The amygdala's response to the stimulus takes place about 100 ms after receiving the stimulus, 100 ms before the neocortex's response which takes place about 200 ms after receiving the stimulus.

The present invention takes advantage of the amygdala's rapidity of response times to express the subconscious without the examinee's awareness. This is because the examinee's potential consciousness can be grasped prior to correction by the neocortex (control under awareness), thereby demonstrating the examinee's aptitude.

In other words, the icons are perceived visually (i.e., with one's eyes) and at the same time stimulate the amygdala.

The stimulation to the amygdala precedes the stimulation to the frontal lobe.

The information that affects the subconscious includes the response of the amygdala (emotion response), in addition to the information from the sensory organs sensed by neocortex (frontal lobe). Since the response by the neocortex differs from the response by the amygdala by 100 ms, there are two types of subconscious, one being affected by the amygdala's response and the other being less affected by the amygdala's response.

Also, icons, even human icons, do not have facial expressions, but are recognized as graphics. Thus, the icons affect the subconscious of the frontal lobe without stimulating emotional system.

The icon examination invokes this frontal lobe subconscious, and the icon application operation is expressed as a response thereto.

The amygdala, which reacts in 100 ms, will less affect the frontal lobe in its immediate response, but the cumulative emotion may affect the frontal lobe, and this difference appears in a manner how the icons are applied. The present invention utilizes the time lag of this response.

This is the theoretical basis of the present invention based on brain science, cognitive science, neuroscience, and the like, and the present invention grasps the examinee's career aptitude and propensity based on this. As described above, according to the present invention, since the examinee's internal and potential consciousness can be expressed through the visual sense, it is possible to grasp the examinee's mental structure in a multifaceted manner. Therefore, the test results can be directly used to evaluate the examinee's personality and aptitude.

According to the visual approach-based aptitude testing system of the second to fourth aspects of the invention the bounding elements may be determined variously or in a simplified manner, so a variety of investigations and analyses can be carried out, ranging from complicated investigations to simple investigations with less burden on the examinee.

According to the visual approach-based aptitude testing system of the fifth to seventh aspects of the invention, since it is also possible to determine the icon relationship, the specific pattern, and the specific icon, it is possible to more accurately grasp the examinee's aptitude and propensity.

According to the visual approach-based aptitude testing system of the eighth and/or ninth aspects of the invention, since the specific pattern and the icon relationship can be determined more accurately, it is more effective for grasping the examinee's aptitude and propensity.

According to the visual approach-based aptitude testing system of the tenth to twelfth aspects of the invention, there are lot of the boundary elements, which increases the number of areas. Therefore, it is possible to more accurately perform relatively complicated investigations and a wide range of investigations.

### BRIEF DESCRIPTION OF THE DRAWINGS

FIG. 1A is a screen showing an embodiment of a check sheet and icons used for the visual approach-based aptitude testing system according to the invention; and FIG. 1B is a figure for explaining FIG. 1A.

FIG. 2A shows a template of lines, corners, areas and contact points described in the check sheet of FIGS. 1A and 1B; FIG. 2B is a conversion table of the template of FIG. 2A; and FIG. 2C is a continuation of FIG. 2B.

FIG. 3A is a code table showing the meaning of each icon and the display when each icon is placed on each line, each contact point, each corner, and in each area; FIG. 3B is a continuation of FIG. 3A; FIG. 3C is a continuation of FIG. 3B; FIG. 3D is a continuation of FIG. 30, FIG. 3E is a continuation of FIG. 3D; FIG. 3F is a continuation of FIG. 3E; FIG. 3G is a continuation of FIG. 3F; FIG. 3H is a continuation of FIG. 3G; FIG. 3I is a continuation of FIG. 3H; FIG. 3J is a continuation of FIG. 3I; FIG. 3K is a continuation of FIG. 3J; and FIG. 3L is a continuation of FIG. 3K.

FIG. 4A shows the meaning of “overlapping” of icons; FIG. 4B is a continuation of FIG. 4A; FIG. 4C is a continuation of FIG. 4B; and FIG. 4D is a continuation of FIG. 4C.

FIG. 5A is a code table (single) of logic comments of the visual approach-based aptitude testing system according to the present invention;

FIG. 5B is a continuation of FIG. 5A; and FIG. 5C is a continuation of FIG. 5B.

FIG. 6 is a code table (overlapping) of logic comments of the visual approach-based aptitude testing system according to the present invention.

FIG. 7A is a master sheet for test analysis by the visual approach-based aptitude testing system according to the present invention; FIG. 7B is a continuation of FIG. 7A; and FIG. 7C is a continuation of FIG. 7B.

FIG. 8 is a master sheet for a headquarters computer of the visual approach-based aptitude testing system according to the present invention.

FIG. 9A shows a calculation logic of a crowded arrangement; FIG. 9B shows a calculation logic of a dispersive arrangement; FIG. 9C shows a calculation logic of a deviative arrangement; FIG. 9D shows a calculation logic of a bottom arrangement; FIG. 9E shows a calculation logic of an upper arrangement; FIG. 9F shows a calculation logic of a protruding arrangement; and FIG. 9G shows a calculation logic of a doll arrangement.

FIG. 10 shows criteria separately provided for determining the details of the doll arrangement, including (A) showing a standard specification and (B) showing examples of samples.

FIG. 11 shows the process specifications of the results by the visual approach-based aptitude testing system according to the present invention.

FIG. 12 is a process flow chart illustrating an example of the visual approach-based aptitude testing system according to the present invention.

FIG. 13, part (A) shows an example of the visual approach-based aptitude testing system according to the present invention, by showing scanner input images of icons applied to the check sheet by an examinee, and FIG. 13 part (B) shows locations of input data from part (A) in a computer.

FIG. 14, part (A) shows another example of the visual approach-based aptitude testing system according to the present invention, by showing scanner input images of icons applied to a check sheet by an examinee, and FIG. 14, part (B) shows locations of input data from part (A) in a computer.

FIG. 15, part (A) shows another example of the visual approach-based aptitude testing system according to the present invention, by showing scanner input images of icons applied to the check sheet by an examinee, and FIG. 15, part (B) shows locations of input data from part (A) in a computer.

FIG. 16, part (A) shows another example of the visual approach-based aptitude testing system according to the present invention, by showing scanner input images of icons applied to the check sheet by an examinee, and FIG. 16, part (B) shows locations of input data from part (A) in a computer.

FIG. 17, part (A) shows another example of the visual approach-based aptitude testing system according to the present invention, by showing scanner input images of icons applied to the check sheet by an examinee, and FIG. 17, part (B) shows locations of input data from part (A) in a computer.

FIG. 18A shows an application embodiment of a crowded arrangement; FIG. 18B shows an application embodiment of a dispersive arrangement; FIG. 18C shows an application embodiment of a balanced arrangement; FIG. 18D shows an application embodiment of a deviative arrangement; FIG. 18E shows an application embodiment of a bottom arrangement; FIG. 18F shows an application embodiment of an upper arrangement; FIG. 18G shows an application embodiment of a top-and-bottom arrangement; FIG. 18H shows an application embodiment of a left-and-right arrangement; FIG. 18I shows an application embodiment of a protruding arrangement; FIG. 18J shows an application embodiment of a doll arrangement; FIG. 18K shows an application embodiment of an overlapping arrangement; FIG. 18L shows an application embodiment of a contact arrangement; and FIG. 18M shows an application embodiment of a close-vicinity arrangement.

FIG. 19A is a screen showing another embodiment of the check sheet used for the visual approach-based aptitude testing system according to the present invention; and FIG. 19B is a figure illustrating a template of lines, corners, areas and contact points described in the check sheet of FIG. 19A.

FIG. 20 is a screen showing another embodiment of the check sheet used for the visual approach-based aptitude testing system according to the present invention.

FIG. 21 is a screen showing another embodiment of the check sheet used for the visual approach-based aptitude testing system according to the present invention.

FIG. 22 is a schematic diagram illustrating a computer (headquarters computer) which can implement the visual approach-based aptitude testing system according to the present invention.

### DETAILED DESCRIPTION

Next, a visual approach-based aptitude testing system according to the present invention will be described in more detail with reference to the drawings showing embodiments of the present invention. For the sake of convenience, the same reference numerals are assigned to portions which perform the same functions, and duplicate descriptions thereof are omitted.

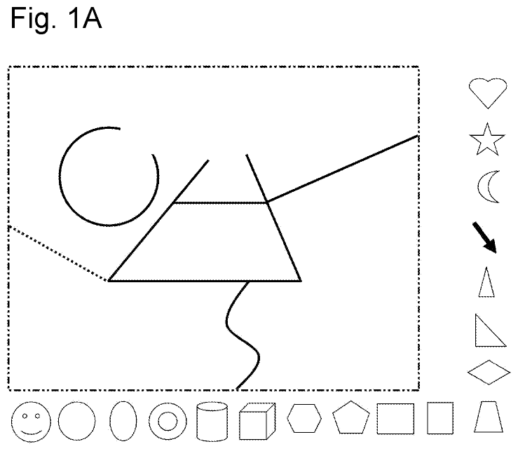

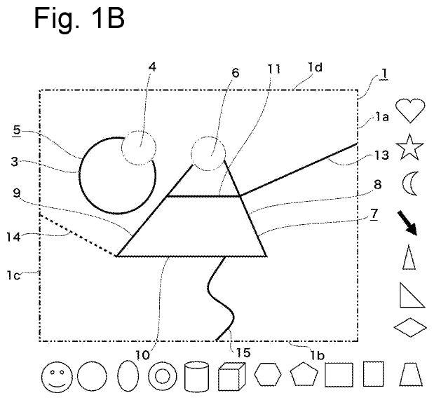

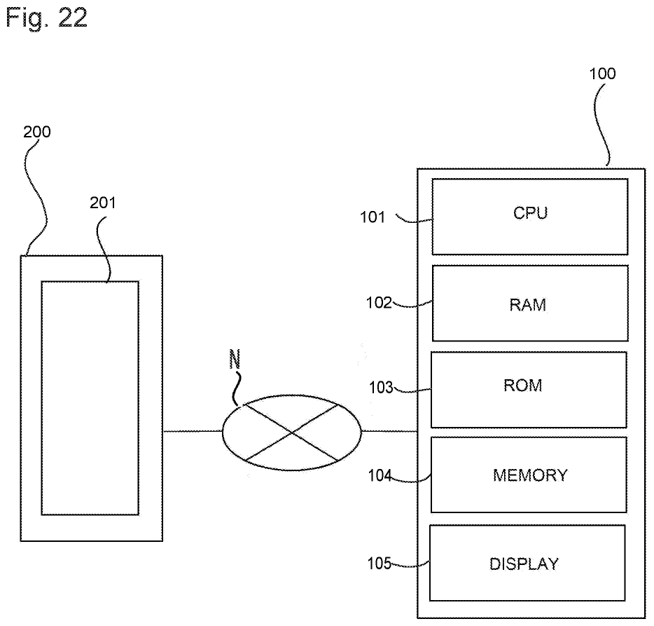

FIGS. 1A and 1B show a check sheet and icons used in a visual approach-based aptitude testing system in accordance with the present invention. In this embodiment, the areas formed by combining the boundary elements (described later) are depicted in a frameless test area, and the check sheet is a so-called open sheet. In this case, the areas shown in a display can be grasped as a window when the check sheet is considered to be a part of a spherical surface. For the sake of convenience, this window is shown by a two-dot chain line in FIGS. 1A, 1B, and the like. The check sheet includes areas comprising a number of circular figures and polygonal figures wherein straight lines, curved lines and broken lines are suitably combined. These form a closed space, an open space, an open circle space, a partly-open trapezoidal space, a slope and a specifically-defined space. An examinee operates his/her portable terminal such as a smartphone 200 with a display 201 (FIG. 22) to apply icons on an image of the check sheet transmitted from a headquarters computer 100 (FIG. 22) via a communication network N. There are 18 types of icons, which will be described later in more detail. The headquarters computer stores the same figures and areas as the check sheet and icons of FIGS. 1A and 1B. The computer 100 includes, for example, a CPU 101 and/or other processor, a RAM 102, a ROM 103 storing a program, other memory 104 (e.g., solid state memory or a hard disk drive), and a display 105. The computer (via the CPU or other processor) executes the program stored in the ROM 103 to perform all of the functions of the headquarters computer described herein. The memory 104, for example, stores information to be transmitted to the examinee's portable terminal to display a display screen including the check sheet and icons shown in FIG. 1A (for example), as well as data in various tables and Figures as described below.

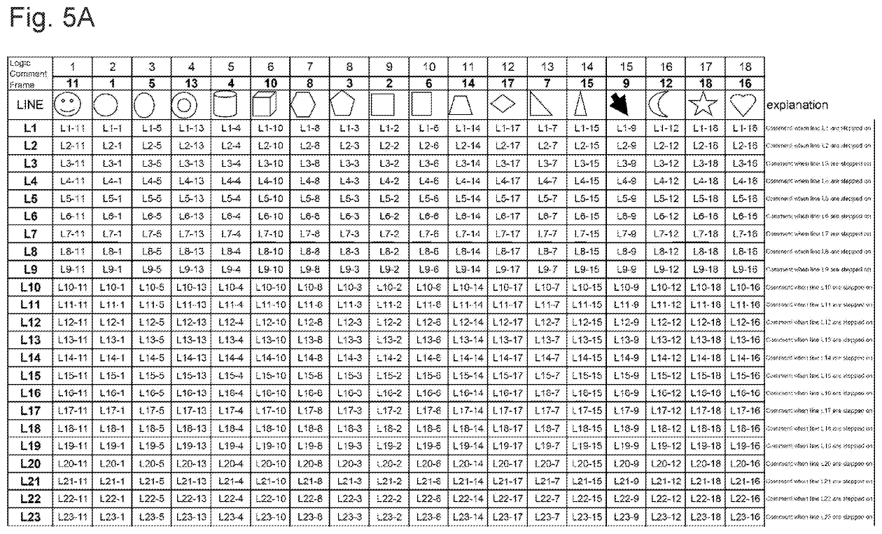

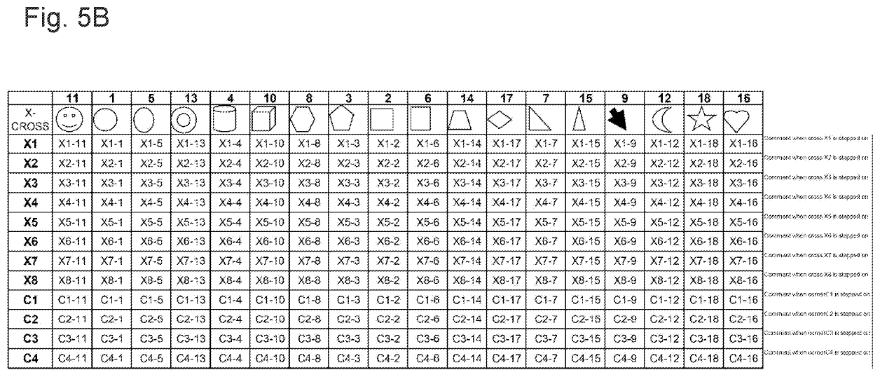

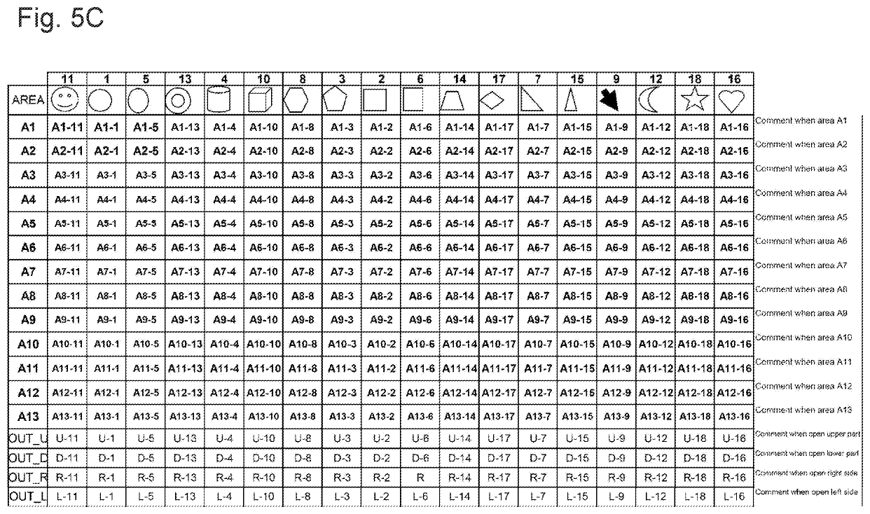

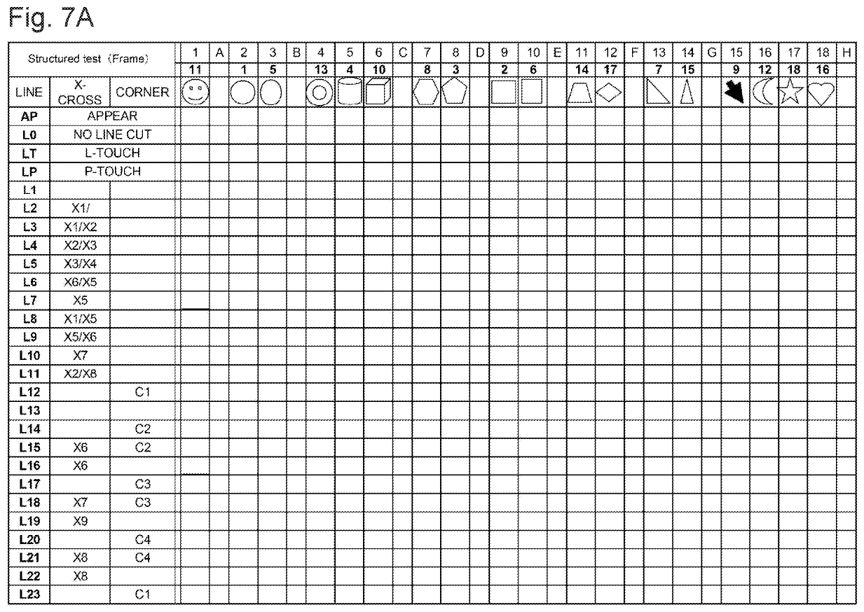

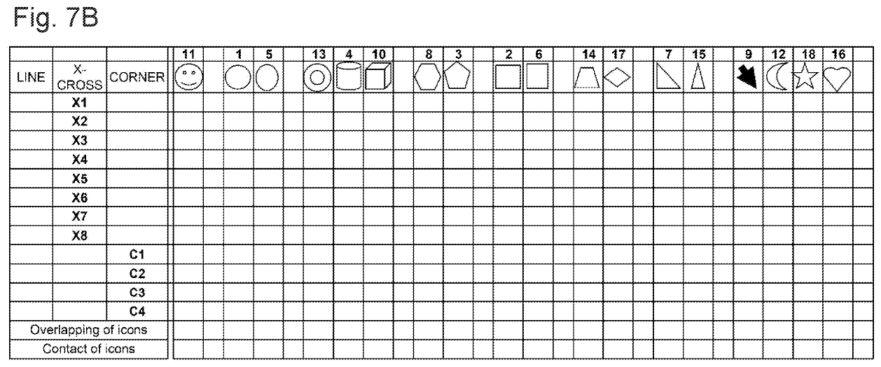

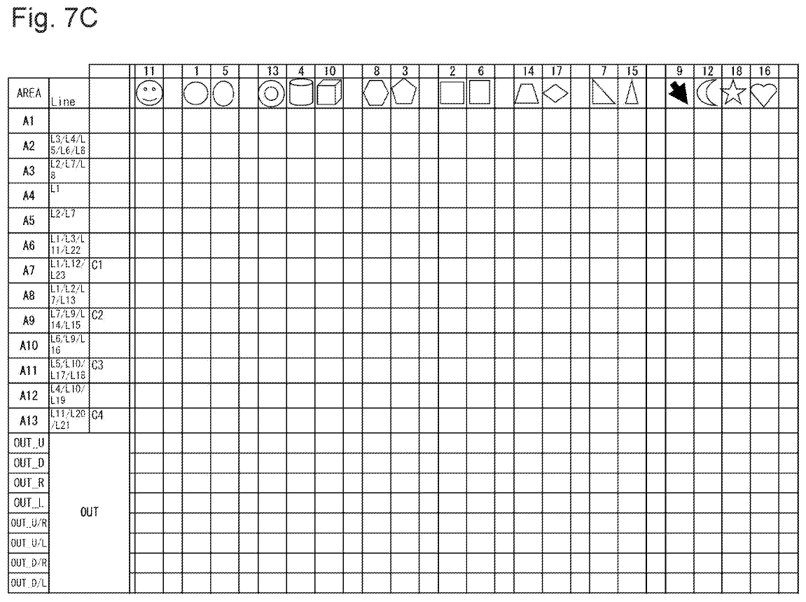

Next, the boundary elements shown in FIGS. 1A and 1B are outlined. More detailed description is given below in the following paragraphs. Different types of the boundary elements are depicted in a window 1 shown by a two-dot chain line, formed by virtual lines 1a-1d (also referred to as window virtual lines), for the sake of convenience. The virtual lines 1a-1d form a virtual frame of the window 1. The boundary elements comprise a circular FIG. 5 having a cut-off 4 at the upper-right part of the circumferential curved line 3, a triangular FIG. 7 having a cut-off 6 at the apex thereof and an intermediate straight line 11 extending in parallel with a straight bottom line 10 between opposite oblique-side lines 8, 9, a straight line 13 (inclined straight line) extending obliquely up and to right from the right end of the intermediate line 11 to the window virtual line 1a, a broken line 14 (inclined broken line) extending obliquely up and to left from the left end (opposite to the inclined straight line 13) of the bottom 10 to the window virtual line 1c, and an S-shape curved line 15 extending down from the rightward middle portion of the bottom 10 to the window virtual line 1b. With these boundary elements, the areas to be described in detail below are divided into nine sectional areas, among which three are located almost in a horizontal direction and the other three almost in a vertical direction. Both cut-offs 4 and 6 open upward. The boundary elements can be provided in any combination and have no inherent meaning (the boundary elements and the combination thereof have no meaning in and of themselves). The boundary elements section the check sheet into many areas as described above and in more detail below.

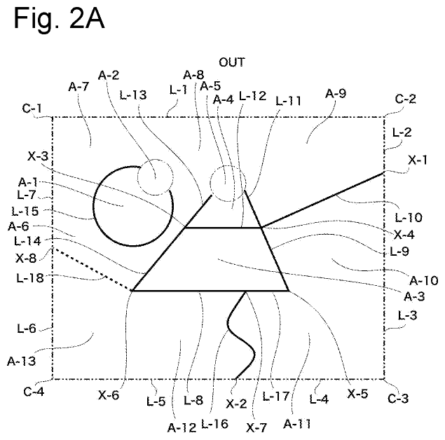

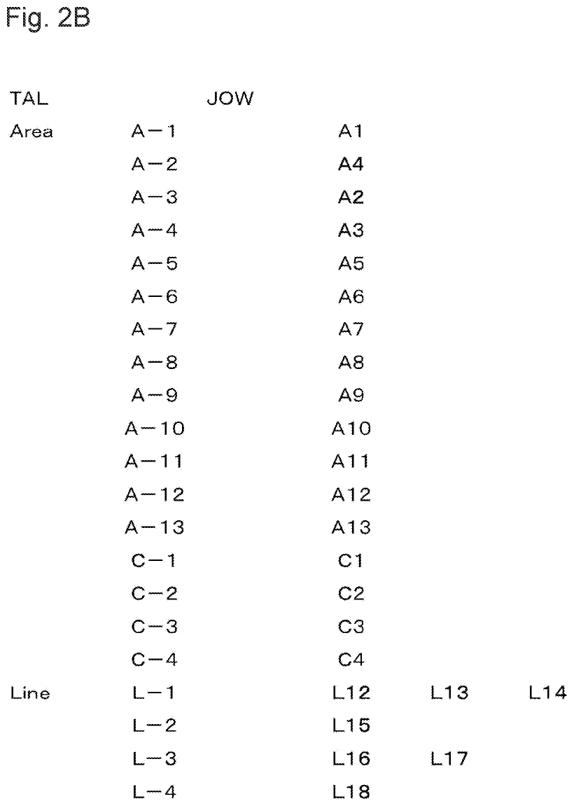

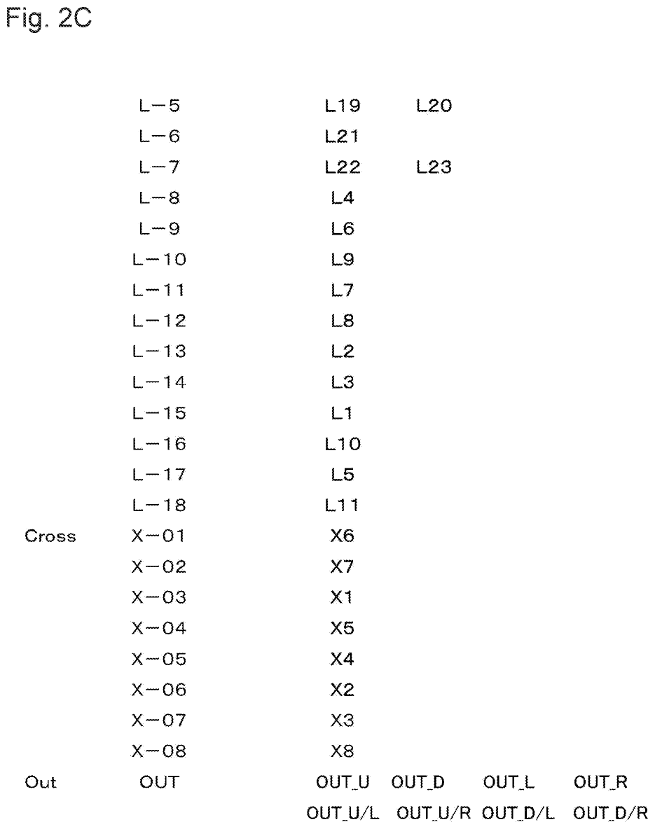

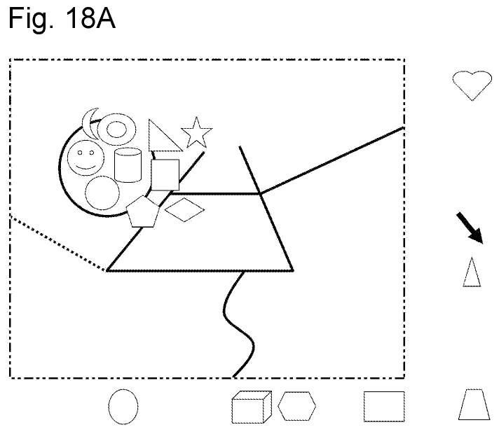

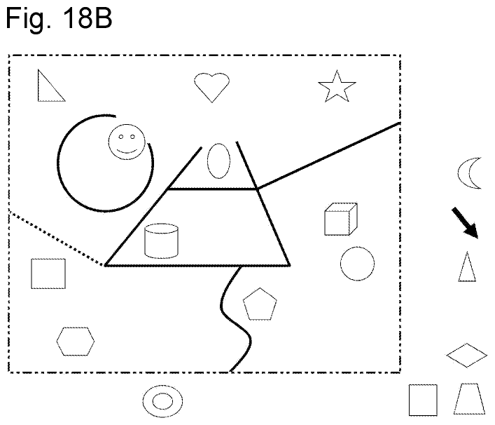

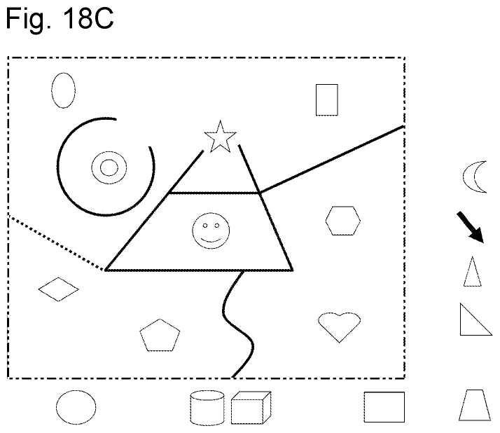

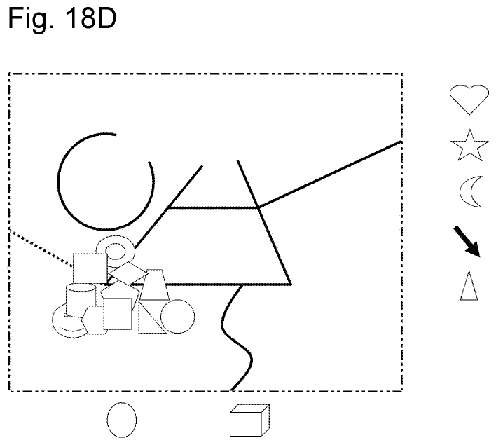

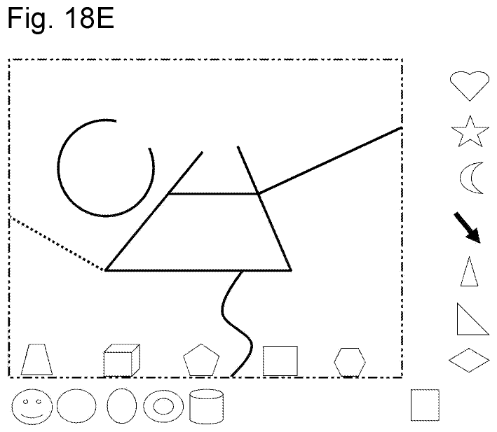

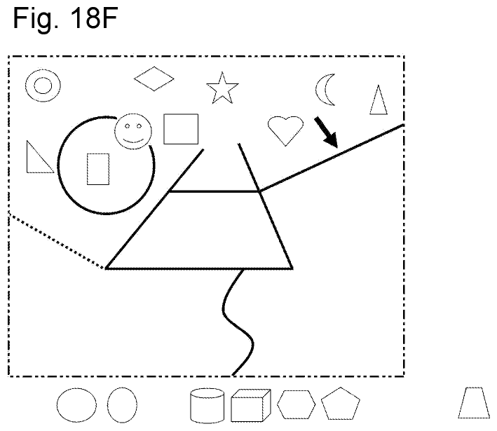

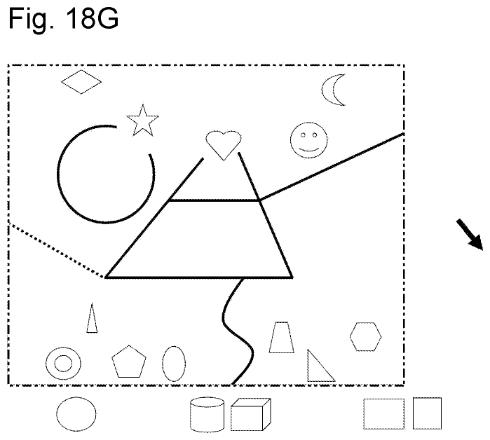

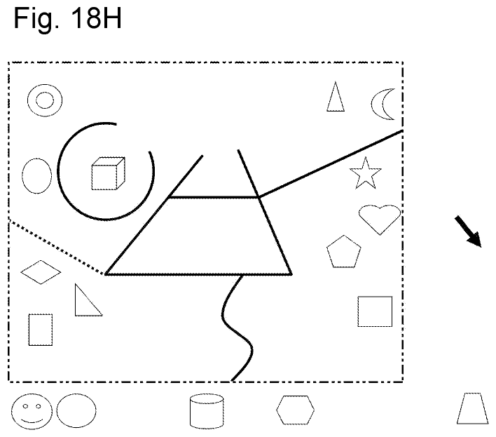

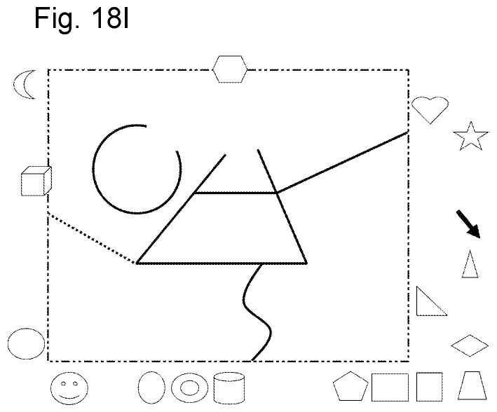

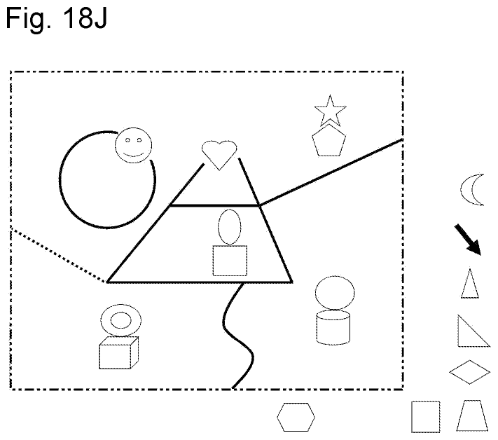

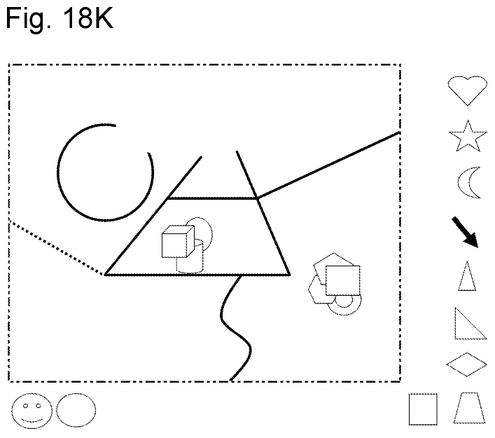

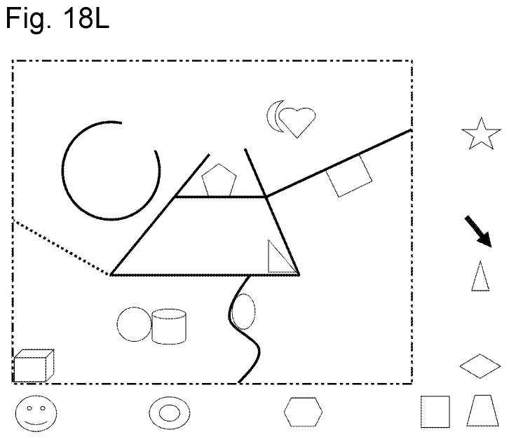

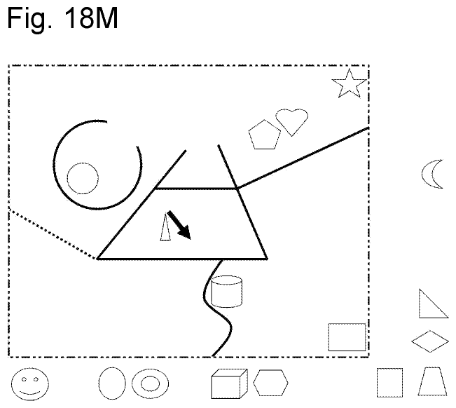

FIG. 2A shows templates of lines, corners, areas and contact points described in the check sheet, and FIGS. 2B and 2C show their conversion tables. TABLE 1A shows lines L1-L18. TABLE 1B shows corners C1-C4. TABLE 1C shows areas A1 to A13. TABLE 1D shows contact points X1-X8. TABLE 2 shows the shape, name and color of each icon. TABLE 3A and TABLE 3B show relationships between icons and their meanings in each of systems A-H listed therein. TABLE 4 shows definitions of meanings for different total numbers of icons selected. TABLE 5 shows definitions of specific patterns (described later) to be grasped in specific situations. TABLE 6 shows definitions of interpretations of these specific patterns. TABLE 7 is a template indicating meanings of icons (described later). Information of FIGS. 1 to 11 (described later), FIGS. 18A to 18M and TABLES 1A to 8 are stored in advance in the memory 104 of the headquarters computer 100.

Here, the contents of TABLES 1A to 8 will be described. TABLE 8 shows relationship among the first table to the sixth table, the individual TABLES, and the drawings. The specified comments in TABLE 3A, TABLE 3B, TABLE 4, TABLE 5 and TABLE 6 are based on the findings statistically obtained from test results.

TABLE 1A

| Line Number | Position of the Line |
|---|---|
| L-1 | A line at the upper side of rectangle chart |
| L-2 | Upper part of the line on the right-hand |
| | side of rectangle chart |
| L-3 | Bottom of the line on the right-hand side |
| | of rectangle chart |
| L-4 | Right of the line at the lower side of |
| | rectangle chart |
| L-5 | Left of the line at the lower side of |
| | rectangle chart |
| L-6 | Bottom of the left-hand side of rectangle |
| | chart |
| L-7 | Upper part of the line on the left-hand |
| | side of rectangle chart |
| L-8 | Left of the line at the bottom of trapezoid |
| | diagram in the center of the chart |
| L-9 | A line on the right-hand side of trapezoid |
| | diagram in the center of the chart |
| L-10 | Line inclined rightward and upward divided |
| | the upper part and bottom of rectangle chart |
| L-11 | A line on the right-hand side of a triangular |
| | diagram drawn at the center of a chart |
| L-12 | A line at the bottom of a triangular diagram |
| | drawn at the center of a chart |
| L-13 | A line on the left-hand side of a triangular |
| | diagram drawn at the center of a chart |
| L-14 | A line on the left-hand side of trapezoid |
| | diagram in the center of the chart |
| L-15 | A line that is an arc of a circle lacking a |
| | part of the circumference drawn on the left |
| | side of the chart |
| L-16 | Line of S curved line that divides the area |
| | A-11 and the area A-12 drawn at the bottom |
| | of the chart |
| L-17 | Right side of the line that forms the bottom |
| | of trapezoid diagram drawn in the center of |
| | the chart |
| L-18 | A line of broken line that divides the upper |
| | part and bottom of the chart on the left side |
| | join the intersection X-6 and the |
| | intersection X-8. |

TABLE 1B

| Corner Number | Position of the Corner |
|---|---|
| C1 | Upper left part |
| C2 | Upper right part |
| C3 | Lower right part |
| C4 | Lower left part |

TABLE 1C

| Area Number | Shape of the Area |
|---|---|
| A-1 | An area consisting of a circle with a missing |
| | upper right corner located in the upper left |
| | corner of the chart |
| A-2 | The part of the missing arc in the area A-1 |
| | (upper right part) |
| A-3 | An area consisting of trapezoid located in |
| | the center of the chart |
| A-4 | Area consisting of a top-missing triangle |
| | connected to the upper part of the A-3 area |
| A-5 | The upper part missing A-4 area |
| A-6 | An area that the upper left part is divided |
| | integrally with the area A-7 and the upper |
| | right part is divided integrally with the |
| | area A-8, a circular arc is formed in a part |
| | of the upper part, and consisting |
| | heteromorphic figure of a broken line which |
| | lower line is inclined |
| A-7 | The area that divided as an integral area of |
| | each area of the areas A-8 and A-9, left |
| | side of the area formed in the upper part of |
| | the chart, and circular arc is located in the |
| | lower right side and lower left part adjoins |
| | A-6 Area. |
| A-8 | The area that divided as an integral area of |
| | each of the areas A-7 and A-9, the center of |
| | the area formed in the upper part of the |
| | chart, and the area A-5 is located in the |
| | lower part. |
| A-9 | The area that divided as an integral area |
| | with the areas A-7 and A-8, the right side |
| | of the area formed in the upper part of the |
| | chart, a line L-10 consisting of a right-up |
| | inclined line is formed in the lower part, |
| | and a line L-11 consisting of a left-up |
| | inclined line is formed in the left side. |
| A-10 | It is the upper part area divided as an |
| | integral area with A-11, a line L-10 |
| | consisting of an inclined line rising to |
| | the right is formed at the upper end, and a |
| | line L-9 consisting of an inclined line |
| | rising to the left is formed at the left end |
| A-11 | It is a lower area divided as an integral |
| | area with A-10, and a line L-16 made of S |
| | curved line is formed at the left end, and a |
| | line L-17 is formed at the upper left end. |
| A-12 | It is a right side area divided as an |
| | integral area with A-13, and a line L-16 |
| | made of S curved line is formed at the |
| | right end portion. |
| A-13 | It is a left side area divided as an |
| | integral area with A-12, and a line L-18 |
| | composed of a broken line is formed at the |
| | upper end. |

TABLE 1D

| Contact | |
|---|---|
| Point Number | Position of the contact point |
| X-1 | Intersection of line L-2 and line L-10 |
| X-2 | Intersection of line L-4 and line L-16 |
| X-3 | Intersection of line L-13 and line L-12 |
| X-4 | Intersection of line L-11 and line L-12 |
| X-5 | Intersection of line L-9 and line L-17 |
| X-6 | Intersection of line L-14 and line L-8 |
| X-7 | Intersection of line L-8 and line L-16 |
| X-8 | Intersection of line L-7 and line L-18 |

TABLE 2

| Icon Number | Shape/Name of the Icon | Color |
|---|---|---|
| 1 | Circle | Pink |
| 2 | Square | Light gray |
| 3 | Regular pentagon | Purple |
| 4 | Column | Green |
| 5 | Ellipse | Light yellow |
| 6 | Rectangle | Light blue |
| 7 | Right triangle | Yellow |
| 8 | Regular hexagon | Orange |
| 9 | Arrow | Dark gray |
| 10 | Cube | Light pink |
| 11 | Face (circle and two eye- | White (gray |
| | like small circles in the | for small |
| | circle and mouth-like | circles only) |
| | concave arcuate surface) | |
| 12 | Moon (arcuate surface) | Gray |
| 13 | Doughnut (concentric | Light yellow |
| | circle surface) | green |
| 14 | Trapezoid | Light blue |
| 15 | Isosceles triangle | Dark pink |
| 16 | Heart shape | Blue |
| 17 | Rhomboid | Ivory |
| 18 | Star | Yellow |

TABLE 3A

| | Number | Icon | Overview of Icons' Meanings | Specified Comment |
|---|---|---|---|---|
| A | | Face system | | |
| 1 | 11 | [symbol] | It represents relationships including | Want to be related to |
| | | | people (self and others). The | people. Have someone |
| | | | inclination of the icons represents | who I want to be |
| | | | the bias of thought, and the reversal | targeted. Cannot ignore |
| | | | represents self-denial or neglect of | people. Have someone |
| | | | others. If it exists in enclosed | important. Have someone |
| | | | space (A3), it represents isolation | dislike. |
| | | | or uniqueness, and contact with other | |
| | | | icons requires a separate meaning | |
| | | | search. | |
| B | | O System | | |
| 2 | 1 | [symbol] | It It shows soft objects. It | Kindness. Warmness. |
| | | | expresses peace and satisfaction, and | Calm. I'm not be |
| | | | it expresses ease of cooperativeness. | nervous. Gentle. |
| | | | Contact with a line of the check | |
| | | | sheet represents a vague request and | |
| | | | sense of security. If it exists in | |
| | | | enclosed space, it somehow expresses | |
| | | | one's feelings of loneliness. | |
| 3 | 5 | [symbol] | It it shows soft objects. It | Soft. Good person. |
| | | | represents peace and satisfaction, | People (humans). Eggs, |
| | | | and expresses flexibility in | lukewarm. Can feel easy. |
| | | | cooperativeness. Contact with a line | |
| | | | of the check sheet represents a vague | |
| | | | request and sense of security. If it | |
| | | | exists in enclosed space, it somehow | |
| | | | represents a feeling of loneliness | |
| | | | that is not just oneself. The | |
| | | | inclination of the icons represents | |
| | | | the instability of thought, and | |
| | | | dependence may be strong. If it is | |
| | | | selected, it has a somewhat unique | |
| | | | aspect. | |
| C | | Three-dimensional system | | |
| 4 | 13 | [symbol] | It shows soft objects. Though it | Strange feeling. With |
| | | | represents peace and satisfaction, | holes. Want to escape. |
| | | | cooperativeness with others is thin | Want to hide. Move. |
| | | | Contact with a line of the check | Car. Run. |
| | | | sheet represents a vague despondency | |
| | | | and sense of security. If it exists | |
| | | | in enclosed space, there may be some | |
| | | | strong claims of loneliness of one's | |
| | | | own. The combination with the icons | |
| | | | (9: arrow) requires a strong sense | |
| | | | of fulfillment. | |
| 5 | 4 | [symbol] | It makes people aware of its strong | Want to enrich. Want |
| | | | presence, but it does not have a very | to be satisfied. |
| | | | deep meaning. This is often the case | Abundant. |
| | | | because of the interesting nature of | |
| | | | the icons. Sometimes it represents | |
| | | | treasure, and sometimes it represents | |
| | | | remaining childhood. Regarding the | |
| | | | inclination of the icons, there is a | |
| | | | sense that insensitive to instability. | |
| | | | In some cases, there may be an | |
| | | | unexpected easy consciousness. | |
| 6 | 10 | [symbol] | If it is single, it represents a | Stable. Don't move. |
| | | | strong presence, and contact with | Want to enter in a box. |
| | | | other icons (7/14) is often simply a | Box. |
| | | | presence. Sometimes, contact with a | |
| | | | line of the check sheet may | |
| | | | represents a strong sense of | |
| | | | stability and mental instability. | |
| | | | If it is selected, it has a somewhat | |
| | | | unique aspect. | |
| D | | Rounded-corner system | | |
| 7 | 8 | [symbol] | Although there are little deep | Someone interested. |
| | | | meanings, contact with other icons | A scary person. |
| | | | has many possibilities, and complex | |
| | | | assessments are required when | |
| | | | evaluating. If it is selected, it | |
| | | | has a somewhat unique aspect. | |
| 8 | 3 | [symbol] | If it is selected, it has a fairly | Medicine. Have a |
| | | | unique aspect. In general, it is | dependence. Want to |
| | | | rarely selected, and it is difficult | rely on. Want to |
| | | | to use in terms of the overall | calm down. |
| | | | configuration. It does not mean much | |
| | | | if it is used as a human body. | |

TABLE 3B

| E | | Square system | | |
|---|---|---|---|---|
| 9 | 2 | [symbol] | Contact with the lines and corners | Same as |
| | | | of the check sheet is desirable to | normal. |
| | | | be strong stability and sense of | |
| | | | security, and does not require a | |
| | | | cluttered atmosphere. | |
| | | | They often prefer organizing and | |
| | | | do not prefer irresponsible | |
| | | | things, but sometimes they do not. | |
| | | | If it is used as the center of a | |
| | | | house (contact with 7), not only | |
| | | | is it a building, but also it is | |
| | | | conscious of it as a house, and | |
| | | | it needs to be paid attention. | |
| 10 | 6 | [symbol] | Contact with the lines or corners | Slightly |
| | | | of the check sheet is desirable | different |
| | | | to be strong stability and sense | from normal. |
| | | | of security, and does not require | |
| | | | a cluttered atmosphere. They | |
| | | | prefer organizing, and often do | |
| | | | not prefer irresponsible things. | |
| | | | If it is used as the center of a | |
| | | | house (contact with 7), not only | |
| | | | is it a building, but also it is | |
| | | | conscious of it as a house, and | |
| | | | it needs to be paid attention. | |
| | | | Sometimes they prefer some | |
| | | | uniqueness, and contact with | |
| | | | arrow (9) needs to be noted. | |
| F | | Rhombic system | | |
| 11 | 14 | [symbol] | Strong stabilization is desired, | Stable. |
| | | | and dependence is often low. | Don't move |
| | | | Reversal represents instability | easily. To |
| | | | and may require request or help. | be motivated. |
| | | | In some cases, contact with (2) | |
| | | | as a house has a different meaning | |
| | | | and it is desirable to family | |
| | | | cohesion. | |
| 12 | 17 | [symbol] | Standard forms represents strong | Picky. |
| | | | instability, but they may be | Unstable. |
| | | | insensitive, the instability and | Don't want |
| | | | may not be problematic. Usually, | to calm down. |
| | | | it is applied in an inclined | |
| | | | manner in many cases, and it | |
| | | | represents stability. Contact | |
| | | | with the icons (7) represents an | |
| | | | inclined house and may represents | |
| | | | instability within the family. | |
| | | | Strong self-assertions are few. | |
| | | | dependence may be strong, and it | |
| | | | need to be satisfied. | |
| G | | Sharp system | | |
| 13 | 7 | [symbol] | The arrangement in the opposite Δ | Dangerous. |
| | | | at 45 degrees slope represents | Tight. Sharp. |
| | | | a strong sense of instability and | dangerous. |
| | | | may indicate an unrest of mind. | Doubtful. |
| | | | In many cases, two-sided contact | |
| | | | at the four corners of the check | |
| | | | sheet has a strong dependence and | |
| | | | is required to be stable and | |
| | | | secure. Contact with icon (2) | |
| | | | represents the house and it needs | |
| | | | to be analyzed as a house or a | |
| | | | family. | |
| 14 | 15 | [symbol] | It may represent sharpness and | Cut. |
| | | | acuteness, and may represent a | Dangerous. |
| | | | strong demand. It has a meaning | Stick. Be |
| | | | as a sword and a meaning as a | stuck. |
| | | | arrow, and it needs to be | A cone. |
| | | | distinguished. In the case of a | |
| | | | sword arrangement, it is necessary | |
| | | | to interpret the meaning by paying | |
| | | | attention to the orientation. In | |
| | | | particular, contact with icons | |
| | | | (11) needs to be paid attention | |
| | | | The same as for icon (1/5). | |
| H | | Unique system | | |
| 15 | 9 | [symbol] | The arrangement as a → is | Attention. |
| | | | primarily, but it also has a | See. Here. |
| | | | mining as a spear. In particular, | Perspective. |
| | | | contact with icons (11) needs to | |
| | | | be paid attention. The same as for | |
| | | | icon (1/5). In addition, this may | |
| | | | mean escape or release from a | |
| | | | specific space, and the | |
| | | | directionality in A1/A3/A4 needs | |
| | | | to be noted. | |
| 16 | 12 | [symbol] | It may be used as a moon simply, | Anxiety. |
| | | | or it may be arranged in a | Scared. |
| | | | structured manner by contact with | Somehow. |
| | | | icon (11). There is no concern if | Vague. |
| | | | it is at the upper part of the | |
| | | | check sheet. If it exists in | |
| | | | A1/A3/A4, it may represent a | |
| | | | strong sense of solitude. | |
| 17 | 18 | [symbol] | If it exists at the upper part of | Hope. Wish. |
| | | | the check sheet, it may indicate | Expectations. |
| | | | hope or pleasure, but if it exists | |
| | | | at the bottom, it may indicate a | |
| | | | sense of hopelessness and ask for | |
| | | | help. If it is placed in A5, it | |
| | | | has a self-assertion, and If it is | |
| | | | placed in A1, it represents a | |
| | | | strong sense of security. In the | |
| | | | case of A3, it represents the | |
| | | | existence as a treasure, and this | |
| | | | is often the assertion that it is | |
| | | | cherished. | |
| 18 | 16 | [symbol] | Originally, it was influenced by | Feeling. |
| | | | color, but the color blue | Like. Kind. |
| | | | represents passive love and likes. | |
| | | | If it exists in A1/A3/A4 itself, | |
| | | | their own secret pleasures and | |
| | | | hopes are existed. The incline of | |
| | | | the icon may indicate distrust or | |
| | | | discomfort, and it must be paid | |
| | | | attention if it exists at the | |
| | | | bottom of the check sheet. | |

TABLE 4

| | Total | | It indicates the number of used icons, |
|---|---|---|---|
| | | | and if the number is large, it |
| | | | indicates positiveness or diversity. |
| | | | Conversely, if it is low, it indicates |
| | | | passiveness or prudence. If icons are |
| | | | used in excess of the standard, |
| | | | attention should be paid because of |
| | | | inattentiveness, strength of self- |
| | | | assertion, etc. If it is less than |
| | | | standard, it tends to be self-denial |
| | | | and to be hidden, it is difficult to |
| | | | communicate. |
| α | A system | | System A is the number of icons when |
| | | | icons are applied to locations other |
| | | | than lines (lines/frames) of the check |
| | | | sheet. This means that the line |
| | | | component of the check sheet is not |
| | | | taken into account. |
| β | O system | | The system 0 is obtained by subtracting |
| | | | the system A from Total, and when it is |
| | | | 0, it indicates that icons do not exist |
| | | | on the line components (lines and |
| | | | frames) of the check sheet. There is |
| | | | also a sense of prudence and inability |
| | | | to overtake the line, but it is not be |
| | | | passive. |
| | Total Number of Icons | | Specified comment |
| Count | 2-5 | Few | I don't want to know |
| | | | about myself. I want to |
| | | | hide. It's bothering. |
| | | | Attention deficit, |
| | | | decreased consciousness |
| | 6-10 | Ordinary | There is no protrusion. |
| | | | It is weak to restriction. |
| | | | Common. |
| | 11-17 | Many | Attention deficit, hot |
| | | | flashes, excessive |
| | | | consciousness, and |
| | | | frisk too mach. |

| | | | |
|---|---|---|---|
| Icon | Overlapping | | There is an positiveness and breaking |
| overlapping | | | power. When the number is large, the |
| and contact | | | degree of diversity is indicated, and |
| | | | the formed figure needs to be examined |
| | | | after evaluating the given meanings. |
| | Contact | | There is positiveness and breaking |
| | | | power, but at the same time there is |
| | | | prudence. When the number is large, |
| | | | there are diversity and spread of |
| | | | thought, and the formed figure needs |
| | | | to be examined after evaluating the |
| | | | given meanings. |
| Line-contact | 1st-contact | | In the case of contact with the lower |
| | | | frame, although there is a strong |
| | | | sense of stability and sense of |
| | | | security, there is a tendency to |
| | | | desire it. If it contacts with lines |
| | | | (lines or corners) other than the |
| | | | frame of the check sheet, it |
| | | | represents dependence, emphasis, and |
| | | | sense of security, and they are sought |
| | | | at the same time. If it contacts with |
| | | | the frame of the upper part, it may |
| | | | indicate anxiety or despondency. |
| | 2nd-contact | | If it contacts with any one of the |
| | | | four corners of lines of the check |
| | | | sheet, it indicates strong stability, |
| | | | sscurity, and it is sought at the |
| | | | same time. Numbers represent its |
| | | | intensity When a line other than the |
| | | | frame is contacted, it prefers |
| | | | surprise, and there is a self- |
| | | | assertion different from the others. |
| | | | The search must be tested including |
| | | | contact between the icons. |
| | Icon No. | [symbol] | It indicates the condition of the |
| | 11/ | | person (the person in question/the |
| | | | person) depending on the location of |
| | | | the existence (details are described |
| | | | separately) |
| | Icon No. | [symbol] | It indicates the state of desire |
| | 18/ | | depending on the location of the |
| | | | existence (details are described |
| | | | separately) |
| | Icon No. | [symbol] | It indicates the state of consciousness |
| | 12/ | | depending on the location of the |
| | | | existence (details are described |
| | | | separately) |
| | Icon No. | [symbol] | It indicates the state of feeling |
| | 16/ | | depending on the location of the |
| | | | existence (details are described |
| | | | separately) |
| | Icon No. | [symbol] | It indicates the state in which the |
| | 9/ | | direction is placed depending on the |
| | | | location of the existence (details are |
| | | | described separately) |
| | Icon No. | [symbol] | It indicates the state of the |
| | 11/12 | | symbolized person (the person in |
| | | | question/the person) depending on the |
| | | | location of the existence (details are |
| | | | described separately) |
| | Icon No. | [symbol] | It indicates the state of the person |
| | 11/15 | | (the person in question/the person) |
| | | | who has a strong relationship with |
| | | | others depending on the location of |
| | | | the existence (details are described |
| | | | separately) |

TABLE 5

| | Specific | | |
|---|---|---|---|
| | Pattern | Definitions | Specified Comment |
| Specific | Crowded | A state that large | Strong dependence. |
| Situation | arrangement | number of icons | Strong picky. |
| | | clustered around | Aggressiveness. |
| | | a specific area | Deflective. Envy |
| | | | jealousy. Cohesion. |
| | | | Can't do something |
| | | | irresponsibly. |
| | Dispersive | A state that icons | Scatter. Distraction |
| | arrangement | are arranged in | of consciousness. |
| | | distributing in all | Can't break out of |
| | | or part of the area | my shell. Unknown |
| | | | anxiety. Can't |
| | | | depend on someone. |
| | Balanced | A state that | Equal Awareness, |
| | arrangement | icons are arranged | Averaging Awareness. |
| | | in a well-balanced | Balance sensation, |
| | | manner in areas or | removing anxiety. |
| | | line components | Anxiety dispersion. |
| | | | Awareness of |
| | | | fragmentation. |
| | Deviative | A state in which | Diffuse feeling. |
| | arrangement | icons are arranged | Doing as I like. |
| | | on specific sides | Slack. Deviation of |
| | | (right and left) | consciousness. |
| | | or parts (top and | Indecision. |
| | | bottom), including | |
| | | top, bottom, left, | |
| | | and right of the | |
| | | check sheet. | |
| | Bottom | A state in which | Stability |
| | arrangement | icons are arranged | orientation. |
| | | in close proximity | Dependence. Seek |
| | | or close proximity | peace of mind. Have |
| | | to the bottom of | confidence. Strong |
| | | the check sheet | awareness of the |
| | | (unbalanced state) | current situation. |
| | | | No change. |
| | Protruding | A state that one or | Excessive |
| | arrangement | more of the icons | consciousness, |
| | | protrude out of the | irresponsible. Hot |
| | | check sheet frame | flashes. Elimination |
| | | (stepping on a line) | of Subjects. Stray. |
| | | | Confusing. |
| | Upper | A state that most | Unstable |
| | arangement | icons are arranged | consciousness. To be |
| | | at the upper part | motivated. Hope. |
| | | (unbalanced state) | Desire. First . . . |
| | | | First-time . . . |
| | Doll | A state that | Human consciousness, |
| | arrangement | Tendency to form a | targets. The person, |
| | | Doll by arranging | this person, that |
| | | icons is seen | person. Doll. |
| | | | Keepsake. Attack |
| | | | target Virtual-Object. |
| | Left-and- | A state that most | Unstable. Segregation |
| | right | icons are arranged | awareness. Escape |
| | Arrangement | on the left and | from the center. |
| | | right sides | Fear of the center. |
| | | (unbalanced state) | |
| | Top-and- | A state that most | Anxiety. Somehow |
| | bottom | icons are arranged | frightened. Unknown |
| | arrangement | at the top part | anxiety. |
| | | and bottom | |
| | | (unbalanced state) | |

TABLE 6

| | Specific | |
|---|---|---|
| | Pattern | Specified Comment |
| Interpretation | Overlapping | Behavior under strong |
| of Specific | | conscious. Abnormal. |
| Situation | | Consciousness |
| On (present)/ | | expression. Biased |
| off (none) | | thought. Excessive |
| | | consciousness. Ignore |
| | | rules. Egocentric. |
| | | Integration |
| | | consciousness. |
| | Contact | Feelings to approach |
| | | of interest. Awareness |
| | | of interval. Dependence. |
| | | Look into empathy. Want |
| | | a dialogue or |
| | | relationship. |
| | Line-contact | Strong dependence. Want |
| | | stability. Escape from |
| | | loneliness. |
| | | Consciousness according |
| | | to rules. Telepathy. |

TABLE 7

| Number | Designation | Item | Definitions | Sample |
|---|---|---|---|---|
| 1 | A | Number | Number of applied icons | |
| | | | (including protruding icons) | |
| 2 | B | [symbol] | If this icon is present, “1” | |
| | | | is input. | |
| 3 | | [symbol] | If this icon is present, “1” | |
| | | | is input. | |
| 4 | | [symbol] | If this icon is present, “1” | |
| | | | is input. | |
| 5 | | [symbol] | If this icon is present, “1” | |
| | | | is input. | |
| 6 | | [symbol] | If this icon is present, “1” | |
| | | | is input. | |
| 7 | | [symbol] | If this icon is present, “1” | |
| | | | is input. | |
| 8 | C | Crowded | A case where many icons are | See FIG. 18A |
| | | Arrangement | gathered (in contact with | |
| | | | each other or overlapped) in | |
| | | | some areas | |
| 9 | | Dispersive | A case where icons are | See FIG. 18B |
| | | Arrangement | applied mostly distributed | |
| 10 | | Balanced | A case where they are applied | See FIG. 18C |
| | | Arrangement | one by one, for example, in | |
| | | | each area in a balanced | |
| | | | manner | |
| 11 | | Deviative | A case where the icons are | See FIG. 18D |
| | | Arrangement | applied in an overlapping | |
| | | | manner so as to form a | |
| | | | cluster. | |
| 12 | | Bottom | A case where three or more | See FIG. 18E |
| | | Arrangement | icons are applied in the | |
| | | | bottom area (including the | |
| | | | bottom line) | |
| 13 | | Upper | A case where three or more | See FIG. 18F |
| | | Arrangement | icons are applied in the | |
| | | | upper area | |
| 14 | | Top-and- | A case where icons are | See FIG. 18G |
| | | Bottom | applied to the top and bottom | |
| | | Arrangement | areas, with no icons in the | |
| | | | middle area (other than the | |
| | | | top and bottom areas) | |
| 15 | | Left-and- | Acase where icons are applied | See FIG. 18H |
| | | Right | to the right and/or left side | |
| | | Arrangement | areas, with no icons in the | |
| | | | middle area (other than the | |
| | | | left and riqht side areas) | |
| 16 | D | Protruding | A case where any icon extends | See FIG. 18I |
| | | Arrangement | beyond the line of the frame, | |
| | | | including a case where there | |
| | | | is any icon stepping on the | |
| | | | line of the frame | |
| 17 | E | Doll | A case where the face and | See FIG. 18J |
| | | Arrangement | circular icons are applied to | |
| | | | specified positions (A4/A5), | |
| | | | and another case where they | |
| | | | are applied to form a doll | |
| | | | (a head and a body) at other | |
| | | | positions | |
| 18 | F | Overlapping | A case where icons are given | See FIG. 18K |
| | | | in an overlapping manner | |
| 19 | | Contact | A case where icons are in | See FIG. 18L |
| | | | line-, face- or point-contact | |
| | | | with each other | |
| 20 | | Close- | A case where three or more | See FIG. 18M |
| | | Vicinity | icons are applied in close | |
| | | | vicinity, nearly in contact | |
| | | | with each other | |

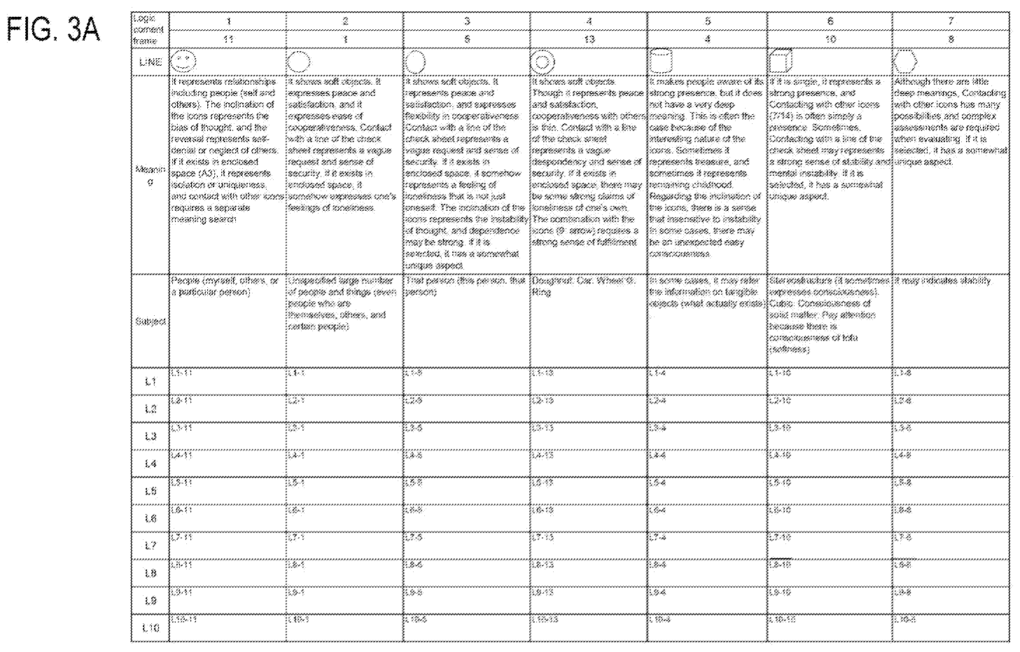

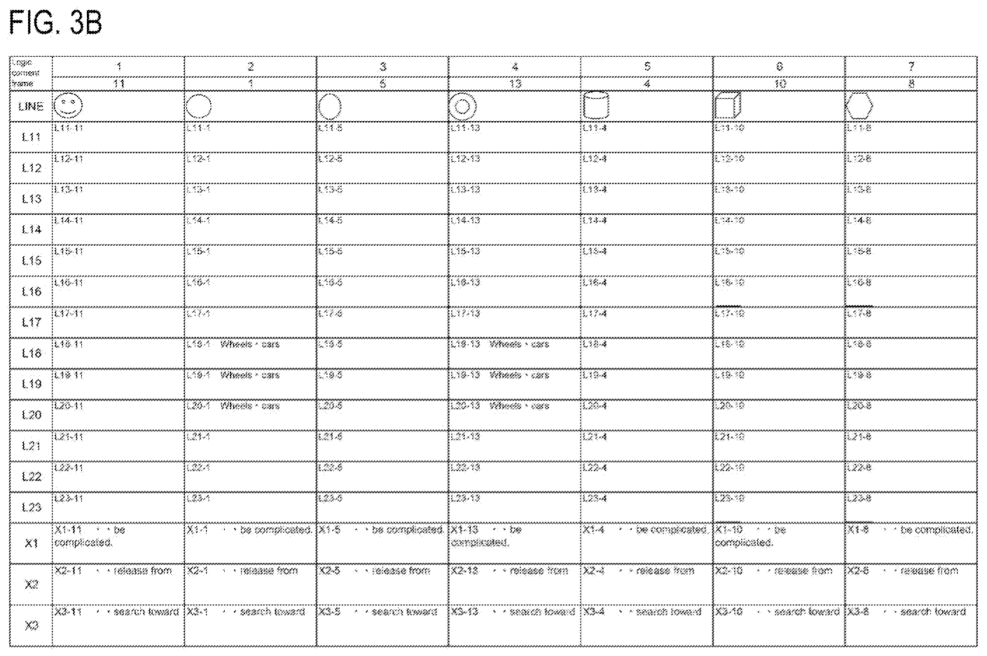

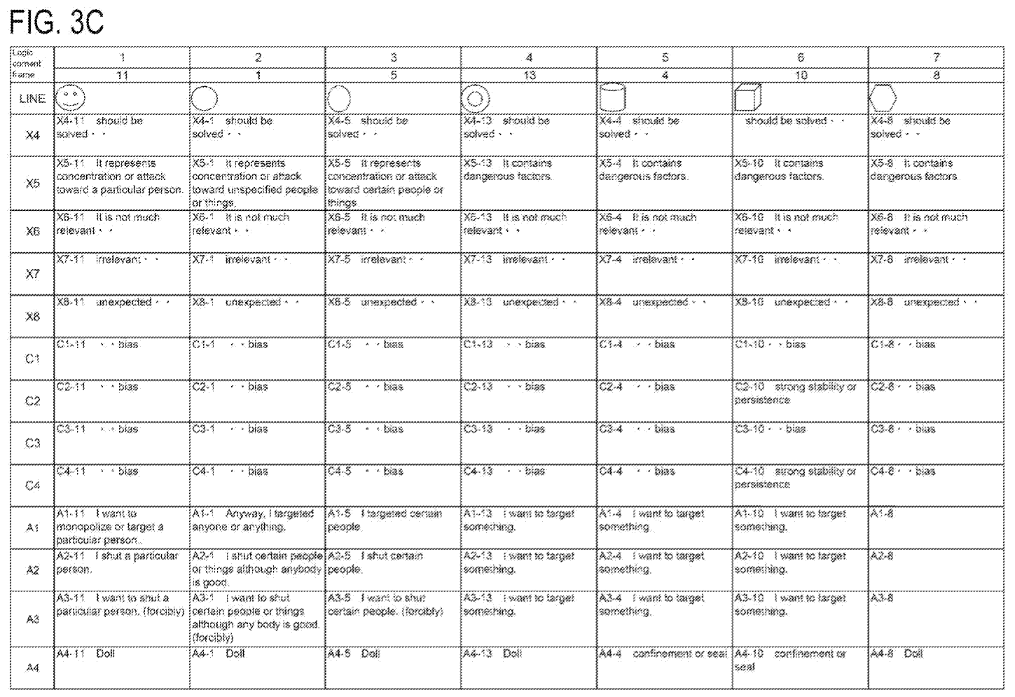

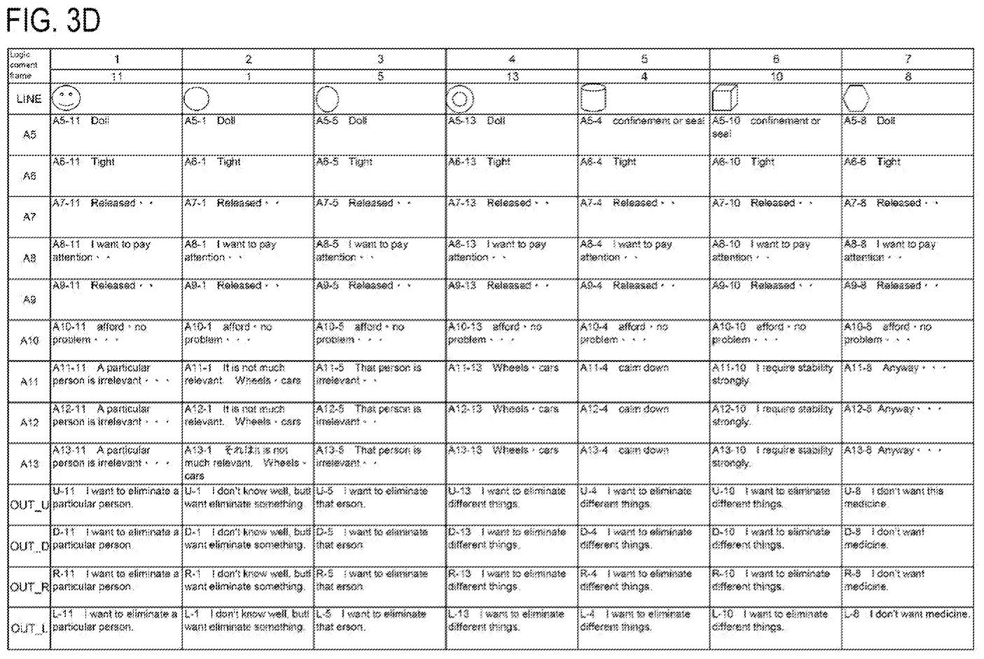

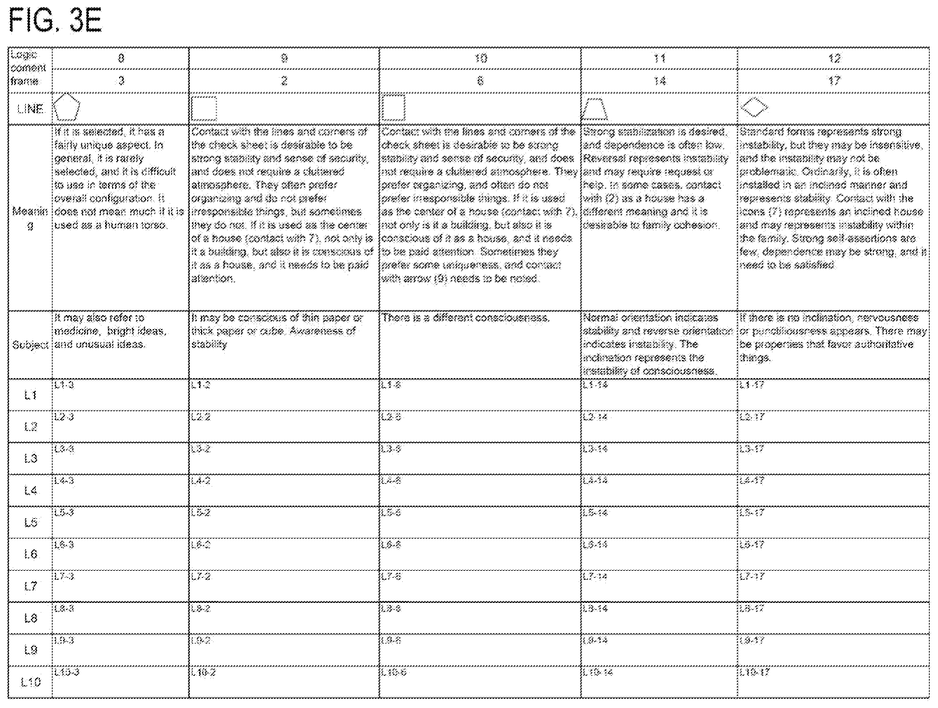

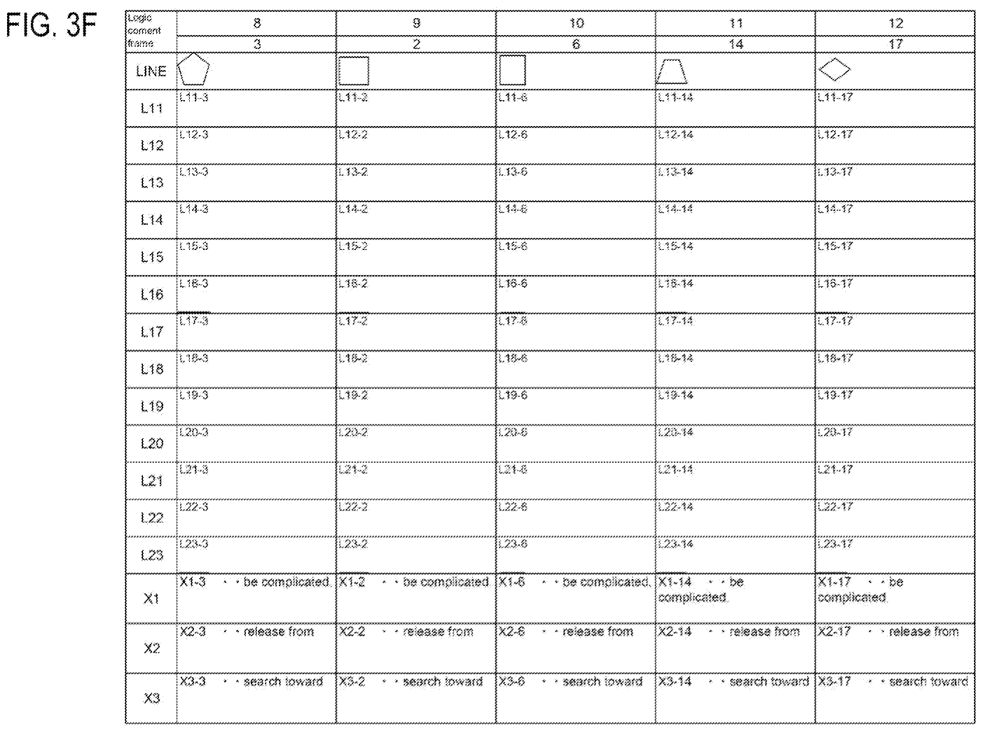

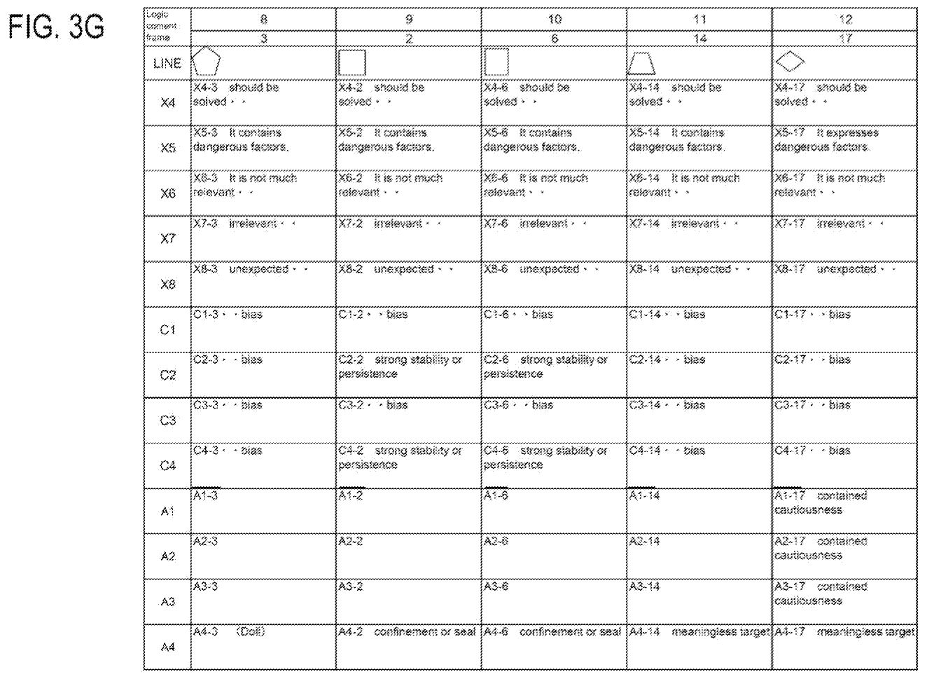

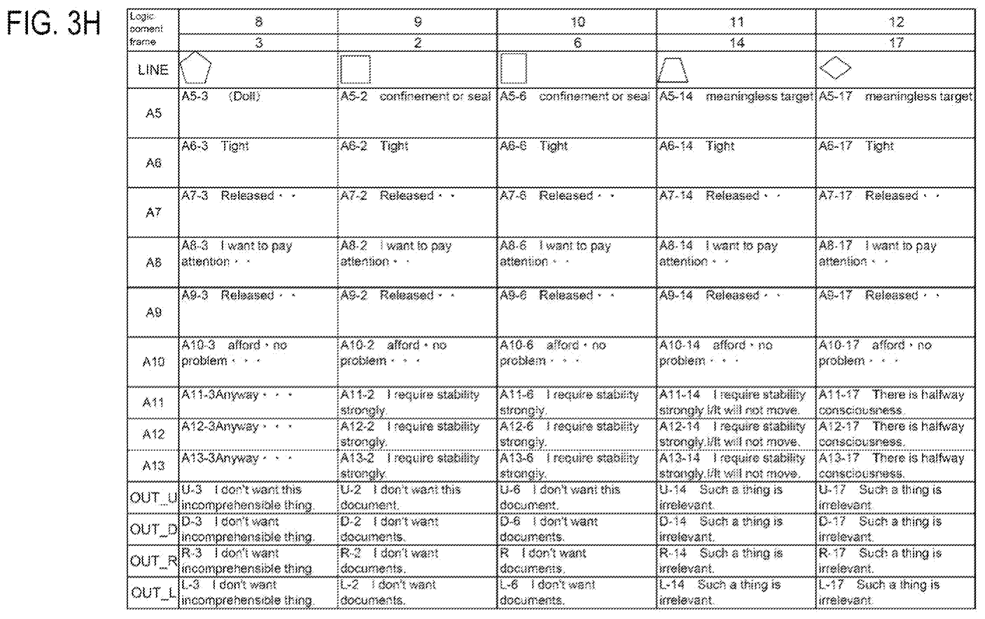

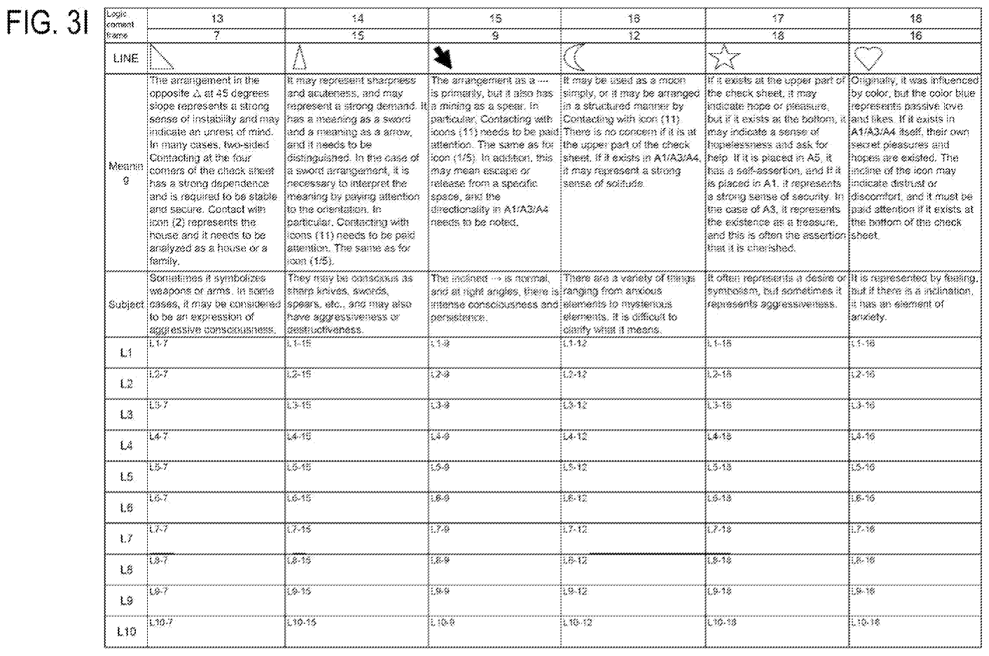

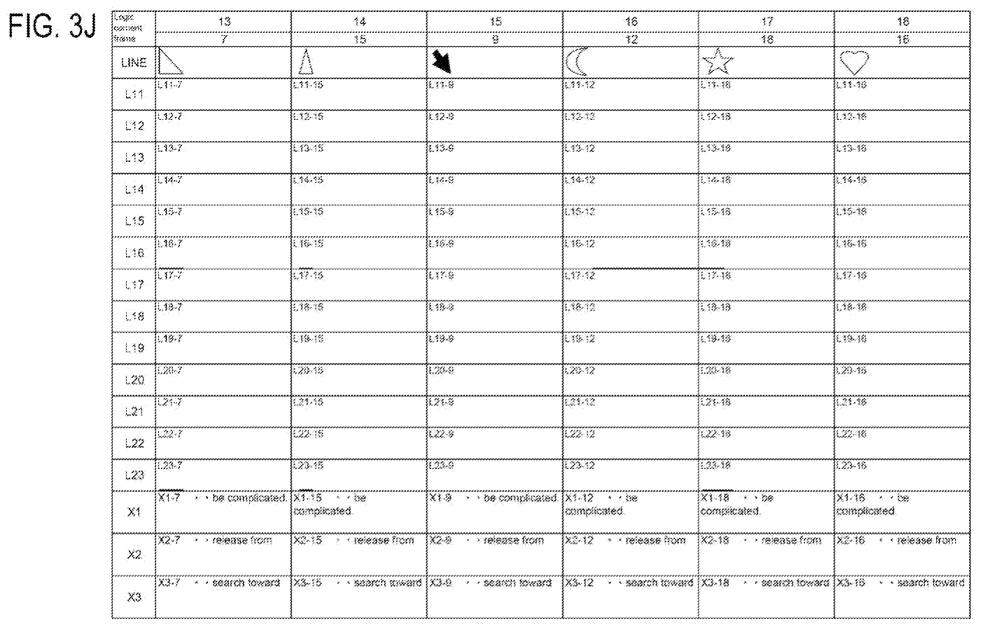

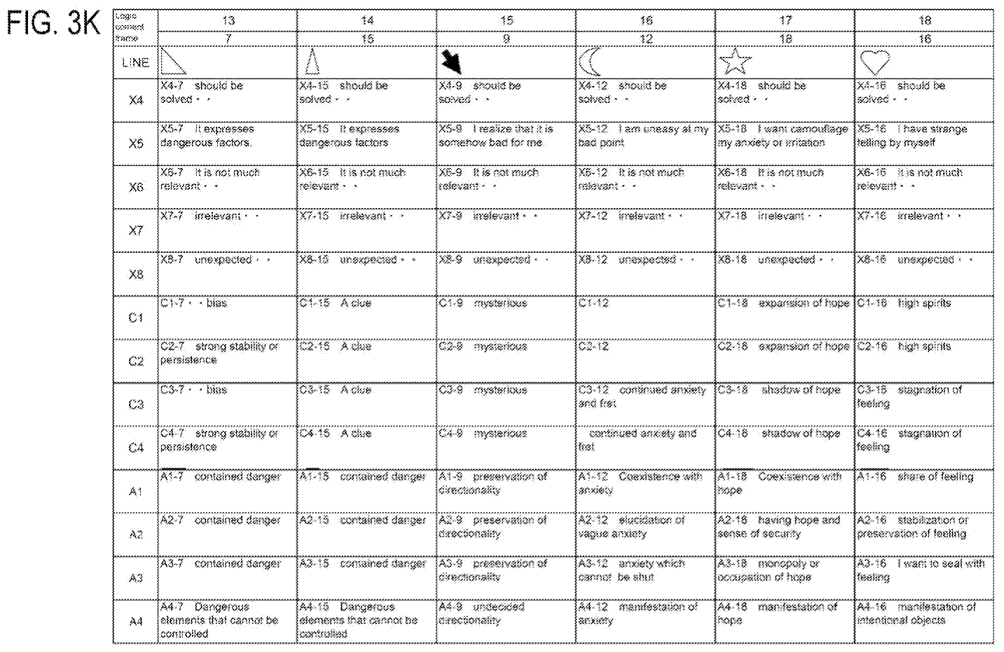

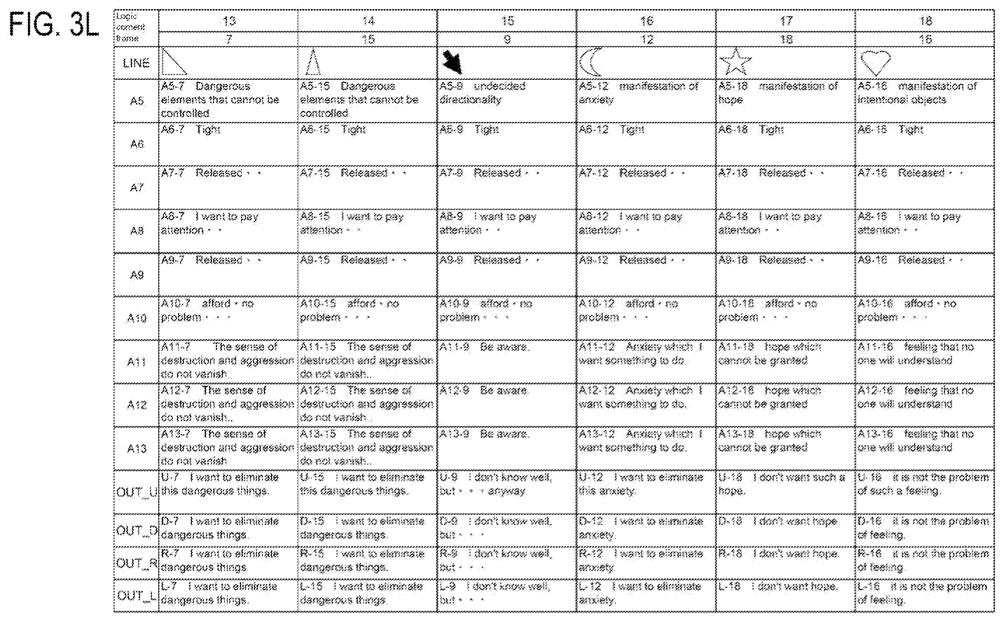

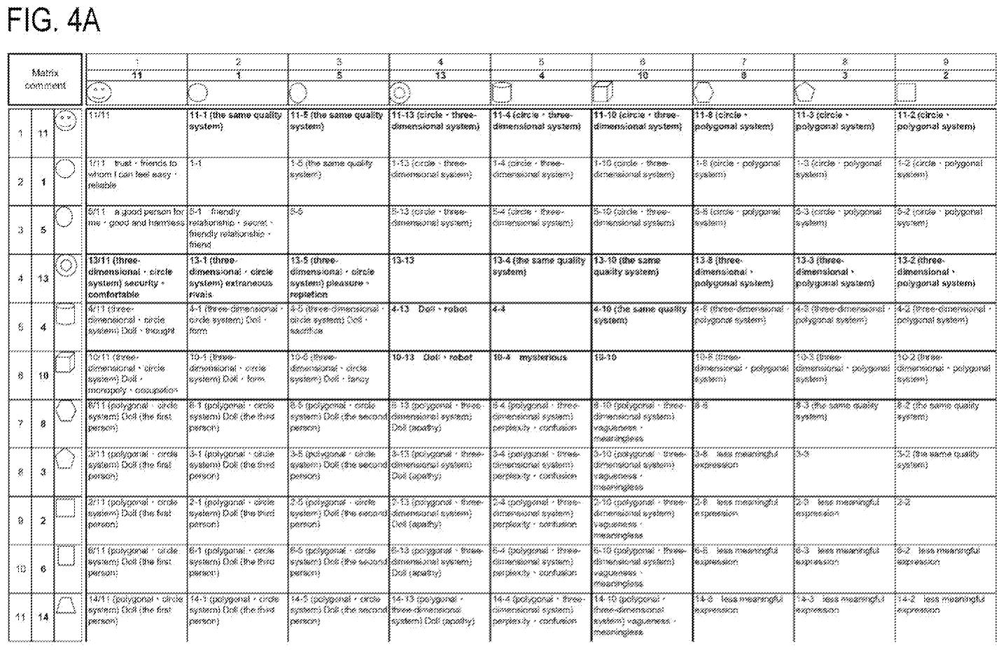

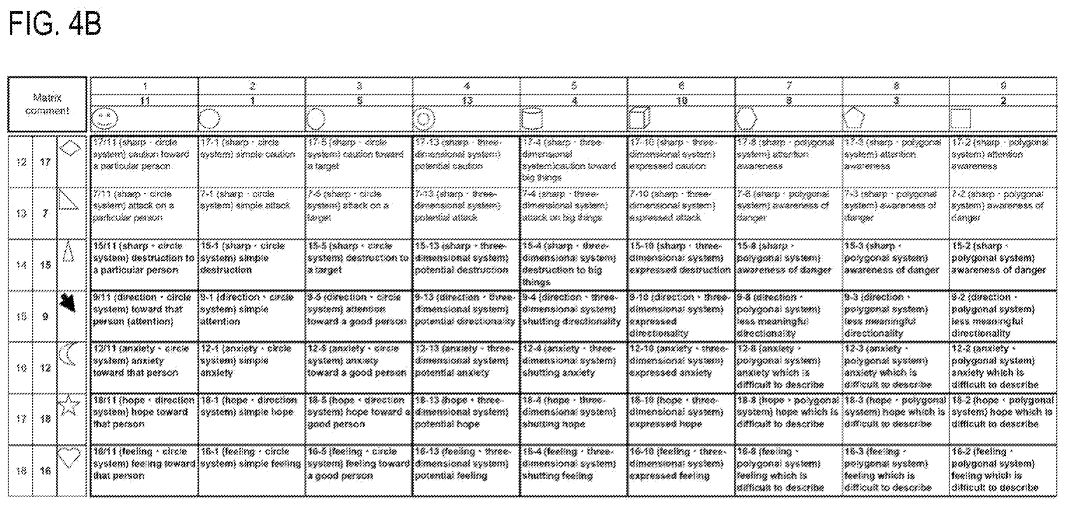

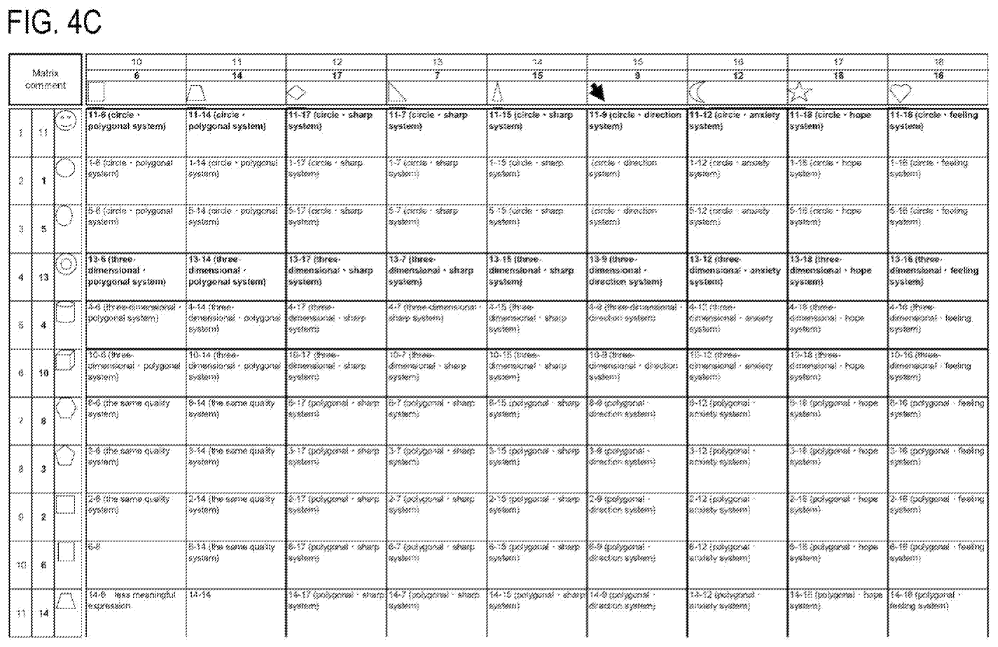

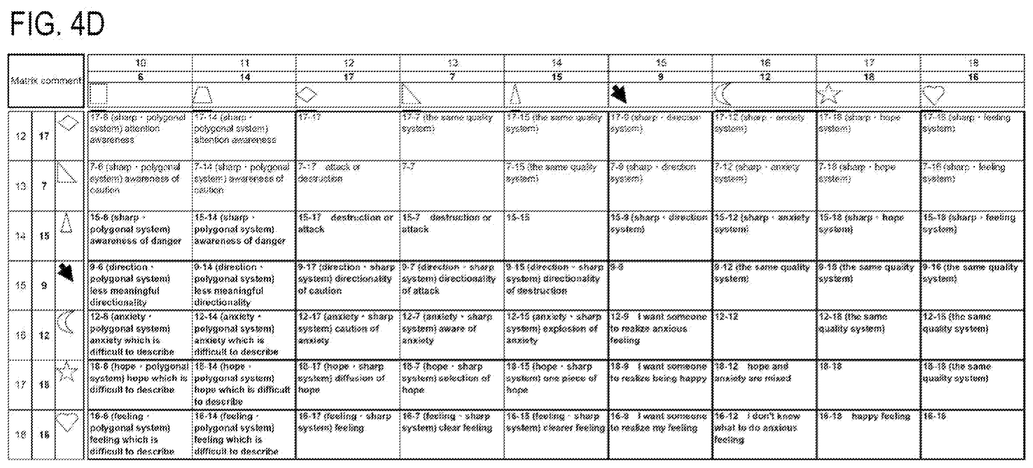

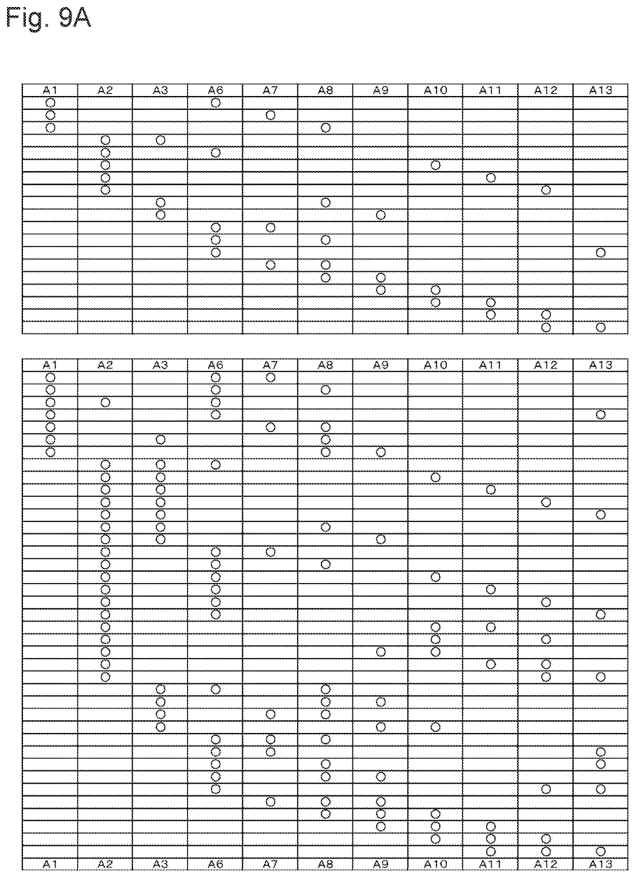

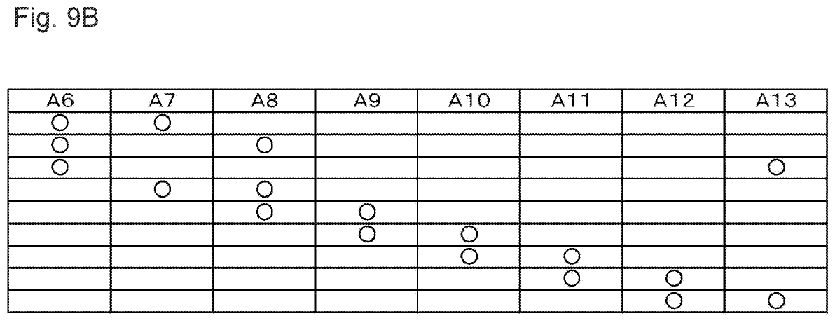

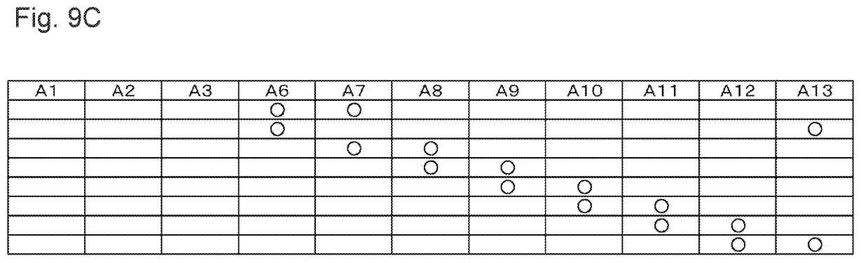

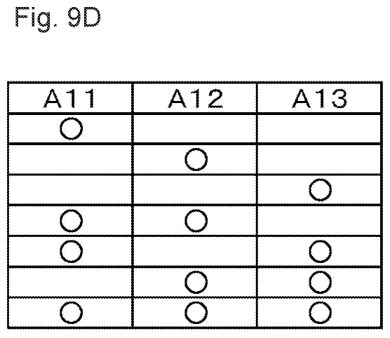

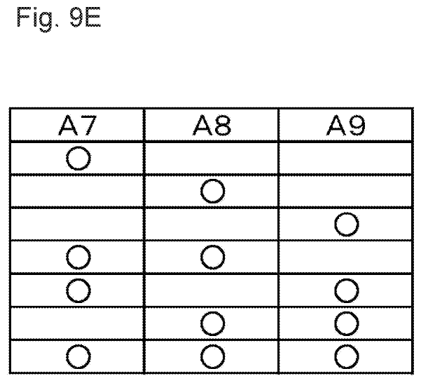

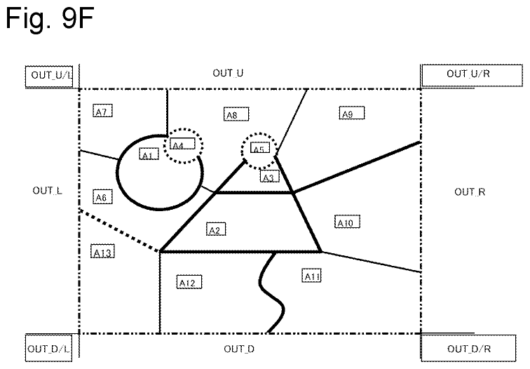

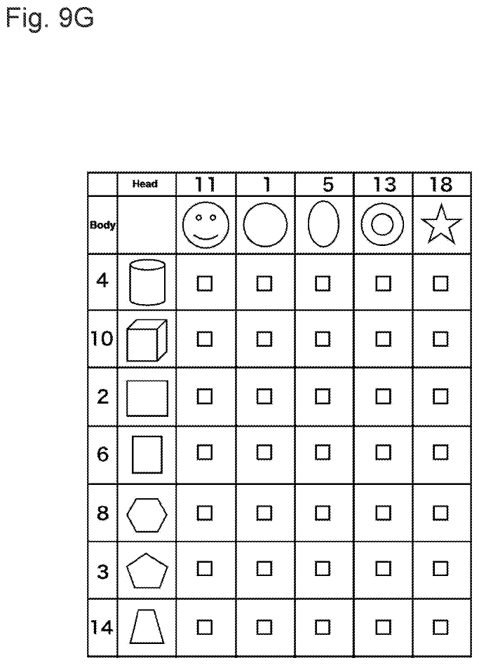

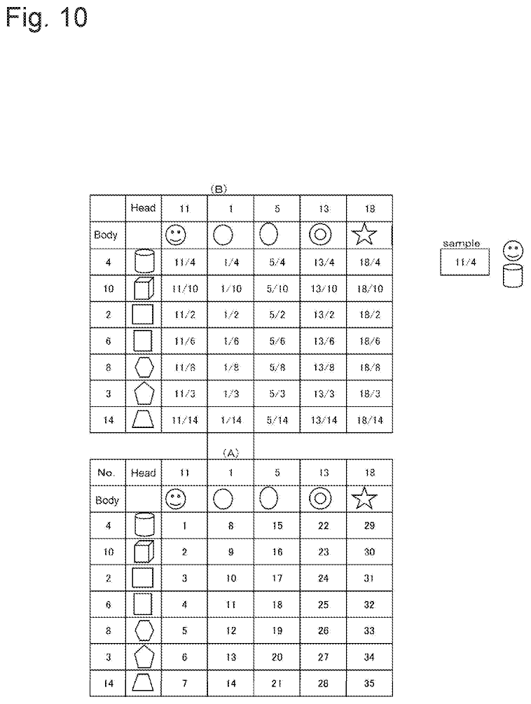

TABLE 8

| table | TABLES and FIGS. | Major Contents |
|---|---|---|
| table 1 | TABLE 1A | Definition of lines |
| | TABLE 1B | Definition of corners |
| | TABLE 1C | Definition of areas |
| | TABLE 1D | Definition of contact points |
| table 2 | TABLE 2 | Definitions of types of the icons |
| | TABLE 3 | Icons' meanings |
| | TABLE 4 | Meaning of the total number of icons |
| | TABLE 7 | Overlapping and contact, etc. |
| table 3 | FIG. 3A to FIG. 3L | Given meaning depending on the position |
| | | of a particular icon in the check sheet |
| table 4 | FIG. 4A to FIG. 4D | Given meaning depending on the |
| | | positional relationship between the icons |
| | | or between the icon and the line (icon |
| | | relationship |
| table 5 | TABLE 5 | Definitions of specific situation and |
| | TABLE 7 | specific patterns (10 types) |
| | FIG. 9A to FIG. 9H | |
| | TABLE 6 | Definitions of specific situation, |
| | | overlapping, “contact, and line-contact |
| table 6 | TABLE 7 | Given meaning by appearance of a |
| | FIG. 9H. | specific icon (doll) |
| | FIG. 10. | |

Icons are selected and applied to the check sheet shown in FIGS. 1A and 1B by the examinee. Icons identical to the icons shown in FIGS. 1A and 1B are stored in the computer 100. In the present invention, the number of icons to be applied by the examinee is limited to 10-15.

The reason for this is that, in the present invention, it is necessary to test a large number of examinees rather than looking at one individual examinee, and therefore, the number of application should be limited. Therefore, in the present invention, the icon size is reduced to the extent that the examinee would not feel frustration even when selecting the lowest number of the icons. In other words, in the present invention, in order for the examinee to feel a degree of freedom when applying the icons to the check sheet, the icons should be relatively small so that they do not occupy a large area of the check sheet when they are applied. This will expand spatial recognition when the icons are applied to the check sheet.

In the memory 104 of the computer 100, the definitions of the lines, areas, corners and contact points described in the check sheet are stored in advance in a first table, and the types of the icons and the definitions of their meanings are stored in a second table.

A third table stored in the memory 104 stores the given meanings/definitions and code numbers as shown in FIGS. 3A to 3L depending on the position of a particular icon in the check sheet, i.e., on a particular line, at a particular corner, in a particular area or at a particular contact point.

A fourth table in the memory 104 stores the given meanings/definitions and code numbers as shown in FIGS. 4A to 4D depending on the positional relationship between the icons or between the icon(s) and the line(s) (icon relationship).

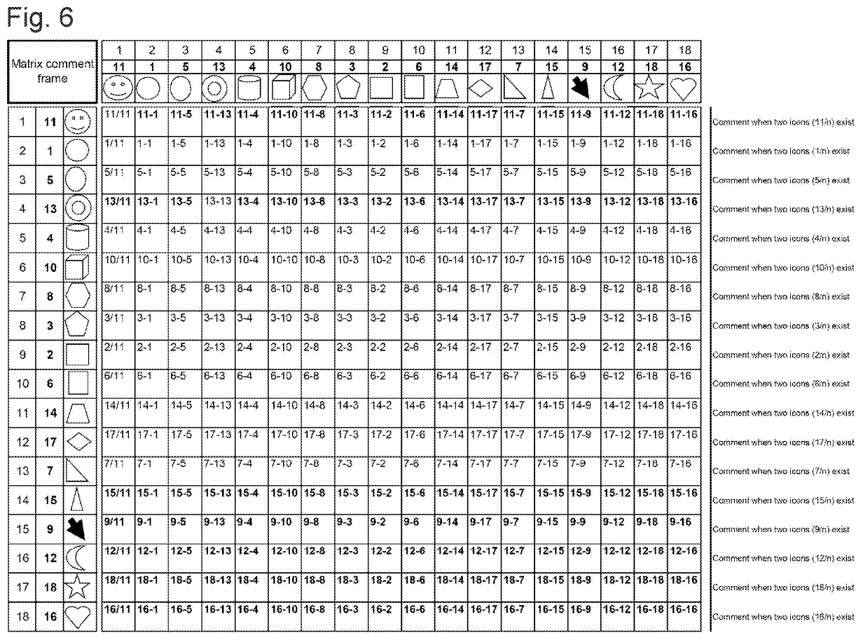

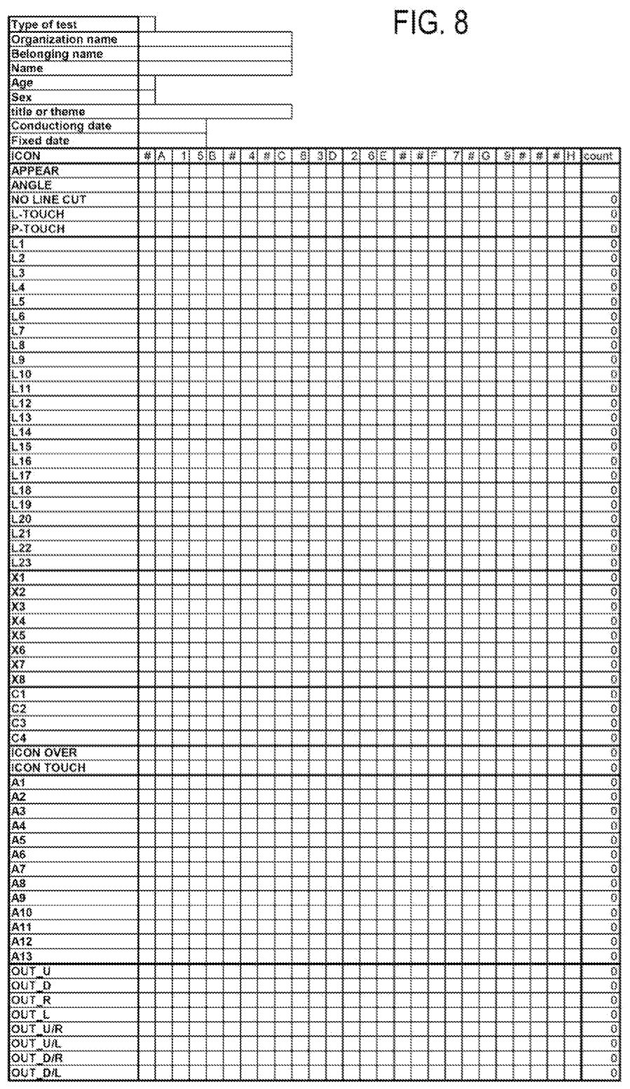

FIGS. 5A to 5C are code tables (single) of the logic comments in the third table, and FIG. 6 is a code table (combination) of the logic comments in the third table. In addition, FIGS. 7A to 7C are master sheets for test analysis, and FIG. 8 is a master sheet for the headquarters computer 100.

A fifth table stored in the memory 104 stores the specific patterns defined in TABLE 5 and TABLE 6 as specific situations. Ten types of specific patterns will be described later with reference to FIGS. 9A to 9G. In addition, the fifth table stores specific patterns defined for “overlapping”, “contact”, and “line-contact” shown in TABLE 6, as well as “close-vicinity” to be described later.

A sixth table stored in the memory 104 stores definitions defined in advance as shown in TABLE 7, FIG. 9G and FIG. 10 with respect to the given meaning in a case wherein a specific icon appears.

Each icon described in TABLE 3A, TABLE 3B, and TABLE 4 is sorted into eight groups as follows and have designated comments stored in the above-described tables.

A system (human system): Face

Want to be related to people. Have someone who I want to be targeted. Cannot ignore people. Have someone important. Have someone dislike.

B system (circular system): circle, ellipse

Circle: kindness. Warmness. Calm. I'm not be nervous. Gentle.

Ellipse: soft. Good person. People (humans). Eggs, lukewarm. Can feel easy.

C system (three-dimensional system): doughnut, column, cube

Doughnut: Strange feeling. With holes. Want to escape. Want to hide. Move. Car. Run.

Column: Want to enrich. Want to be satisfied. Abundant.

Cube: Stable. Don't move. Want to enter in a box. Box.

D system (rounded-corner system): regular hexagon, regular pentagon

Regular hexagon: Someone interested. A scary person.

Regular pentagon: Medicine. Have a dependence. Want to rely on. Want to calm down.

E system (square system): rectangle, square

Rectangle: Same as normal.

Square: slightly different from normal.

F system (rhomboid system): trapezoid, rhomboid

Trapezoid: Stable. Don't move easily. To be motivated.

Rhomboid: Picky. Unstable. Don't want to calm down.

G system (Sharp system): right triangle, isosceles triangle

Right triangle: dangerous. Tight. Sharp, dangerous. Doubtful.

Isosceles triangle: Cut. Dangerous. Stick. Be stuck. A cone.

H system (unique system): arrow, moon, star, heart shape

Arrow: Attention. See. Here. Perspective.

Moon: Anxiety. Scared. Somehow. Vague.

Star: Hope. Wish. Expectations.

Heart shape: Feeling. like. Kind.

In addition, “specific situation” shown in TABLE 5 defines ten types of specific patterns in this embodiment, as described below.

FIGS. 9A to 9G are the specifications for the specific situations in the fifth table. FIG. 9A shows the calculation logic of the “crowded arrangement”, FIG. 9B shows the calculation logic of the “dispersive arrangement”, FIG. 9C shows the calculation logic of the “deviative arrangement”, FIG. 9D shows the calculation logic of the “bottom arrangement”, FIG. 9E shows the calculation logic of the “upper arrangement”, FIG. 9F shows the calculation logic of the “protruding arrangement”, and FIG. 9G shows the calculation logic of the “doll arrangement”.

The definitions and calculation logic of the specific situations are as follows.

The crowded arrangement refers to a case where a centroid of all the icons is located in two or three proximate areas. For example, it is in the areas A2, A10 and A9.

The calculation logic is as follows.

(A) The area A4 is determined as a part of either the area A1 or the area A8. In addition, the area A5 is determined as a part of either the area A3 or the area A8.

(B) A case where there is any icon not belonging to the areas A1-A13 will not be regarded as the “crowded arrangement”.

The proximate areas have following 58 combinations.

<1> A1 and A6, <2> A1 and A7, <3> A1 and A8, <4> A2 and A3, <5> A2 and A6, <6> A2 and A10, <7> A2 and A11, <8> A2 and A12, <9> A3 and A8 <10> A3 and A9, <11> A6 and A7, <12> A6 and A8, <13> A6 and A13, <14> A7 and A8, <15> A8 and A9, <16> A9 and A10, <17> A10 and A11 <18> A11 and A12<19> A12 and A13, <20> A1, A6 and A7, <21> A1, A6 and A8, <22> A1, A2 and A6, <23> A1, A6 and A13, <24> A1, A7 and A8, <25> A1, A3 and A8, <26> A1, A8 and A9, <27> A2, A3 and A6, <28> A2, A3 and A10, <29> A2, A3 and A11, <30> A2, A3 and A12, <31> A2, A3 and A13, <32> A2, A3 and A8, <33> A2, A3 and A9, <34> A2, A6 and A7, <35> A2, A6 and A8, <36> A2, A6 and A10, <37> A2, A6 and A11, <38> A2, A6 and A12, <39> A2, A6 and A13, <40> A2, A10 and A11, <41> A2, A10 and A12, <42> A2, A9 and A10, <43> A2, A11 and A12, <44> A2, A12 and A13, <45> A3, A6 and A8, <46> A3, A8 and A9, <47> A3, A7 and A8, <48> A3, A9 and A10, <49> A6, A7 and A8, <50> A6, A7 and A13, <51> A6, A8 and A13, <52> A6, A8 and A9, <53> A6, A12 and A13, <54> A7, A8 and A9, <55> A8, A9 and A10, <56> A9, A10 and A11, <57> A10, A11 and A12, <58> A11, A12 and A13. (FIG. 9A).

The dispersive arrangement refers to a case where one or two icons are located in an area not in vicinity to the lines. In this case, areas A1, A2 and A3 are not targets of proximate lines.

The calculation logic is as follows.

(A) In a case where two or more icons exist in any one of areas A1, A2 and A3,

(B) In a case where there is any icon not belonging to areas A1-A13,

(C) A case where three or more icons exist in any one of areas A4 to A13 will not be regarded as the dispersive arrangement.

(D) It does not fall under the dispersive arrangement, if <1> there is at least one icon belonging to Area A6 and A7, <2> there is at least one icon in A6 and A8, <3> there is at least one icon in A6 and A13, <4> there is at least one icon in A7 and A8, <5> there is at least one icon in A8 and A9, <6> there is at least one icon in A9 and A10, <7> there is at least one icon in A10 and A11, <8> there is at least one icon in A11 and A12, <9> there is at least one icon in A12 and A13 (FIG. 9B).

The balanced arrangement refers to a case where there is only one icon in one area.

The calculation logic is as follows. The following cases will not be regarded as the “balanced arrangement”.

<1> If there are icons not belonging to areas A1 to A13,
<2> If two or more icons exist in one area,
<3> If the icons exist which number is less or more than the prescribed numbers.

The deviative arrangement refers to a case where all icons exist in the outermost two areas. However, areas A4 and A5 should be considered as area A8. The calculation logic is as follows. The following cases should be regarded as the deviative arrangement. (FIG. 9C)

<1> All icons exist in areas A6 and A7
<2> All icons exist in areas A6 and A13
<3> All icons exist in areas A7 and A8
<4> All icons exist in areas A8 and A9
<5> All icons exist in areas A9 and A10
<6> All icons exist in areas A10 and A11
<7> All icons exist in areas A11 and A12
<8> All icons exist in areas A12 and A13

The bottom arrangement refers to a case where all icons exist in the bottom areas A11, A12 and A13. Therefore, the bottom arrangement includes the following seven cases by this calculation logic (FIG. 9D).

<1> area A11
<2> area A12
<3> area A13
<4> areas A11 and A12
<5> areas A11 and A13
<6> areas A12 and A13
<7> areas A11, A12 and A13

The upper arrangement refers to a case where all icons exist in the upper areas A7, A8 and A9. Therefore, the upper arrangement includes the following seven cases by this calculation logic (FIG. 9E).

<1> area A7
<2> area A8
<3> area A9
<4> areas A7 and A8
<5> areas A7 and A9
<6> areas A8 and A9
<7> areas A7, A8 and A9

The protruding arrangement refers to a case where all icons are stepping on or protruding outward from any of the outer peripheral lines L-1 to L-7. The calculation logic for the protruding arrangement is determined not based on the centroid of the icons but based on the area and line data from the analysis engines. If the flags of the icons in the following eight areas shown in FIG. 9F are all ON, it is regarded as the “protruding arrangement”.

### <1> OUT U/L

### <2> OUT U

### <3> OUT U/R

### <4> OUT R

### <5> OUT D/R

### <6> OUT D

### <7> OUT D/L

### <8> OUT L.

The top-and-bottom arrangement refers to a case where all icons exist in the upper areas A-7, A-8 and A-9 and in the lower areas A-13, A-12 and A-11, which is the calculation logic therefor.

The left-and-right arrangement refers to a case where all icons exist in the left areas A-7, A-6 and A-13 and in the right areas A-9, A-10 and A-11, which is the calculation logic therefor.

The doll arrangement refers to a case where some face icon exists in A4 and/or A5 to form a doll. The calculation logic for the doll arrangement is as follows. First, the icons which may form a face include:

Face (Icon No. 11),

Circle (Icon No. 1),

Ellipse (Icon No. 5),

Doughnut (Icon No. 13),

Star (Icon No. 18).

In addition, it is recognized as the doll arrangement if the body icon(s) is combined with the face icon in the following manners (FIG. 9G).

<1> If the face icon (Icon No. 11) is combined with column (Icon No. 4), cube (Icon No. 10), square (Icon No. 2), rectangle (Icon No. 6), regular hexagon (Icon No. 8), regular pentagon (Icon No. 3) or trapezoid (Icon No. 14)
<2> If the circle icon (Icon No. 1) combined with column (Icon No. 4), cube (Icon No. 10), square (Icon No. 2), rectangle (Icon No. 6), regular hexagon (Icon No. 8), regular pentagon (Icon No. 3) or trapezoid (Icon No. 14)
<3> If the ellipse icon (Icon No. 5) combined with column (Icon No. 4), cube (Icon No. 10), square (Icon No. 2), rectangle (Icon No. 6), regular hexagon (Icon No. 8), regular pentagon (Icon No. 3) or trapezoid (Icon No. 14)
<4> If the doughnut icon (Icon No. 13) combined with column (Icon No. 4), cube (Icon No. 10), square (Icon No. 2), rectangle (Icon No. 6), regular hexagon (Icon No. 8), regular pentagon (Icon No. 3) or trapezoid (Icon No. 14)
<5> If the star Icon (Icon No. 18) combined with column (Icon No. 4), cube (Icon No. 10), square (Icon No. 2), rectangle (Icon No. 6), regular hexagon (Icon No. 8), regular pentagon (Icon No. 3) or trapezoid (Icon No. 14)

FIG. 10 shows criteria separately provided for determining the details of the doll arrangement. This is also stored in the computer's memory 104.

Next, “overlapping”, “contact”, “line-contact” and “close-vicinity” described in TABLE 6 will be described. “Overlapping” refers to a situation in which icons overlap each other, and “contact” refers to a situation in which the peripheral parts of the icons contact each other, including “line-contact” meaning a situation in which the peripheral part of the icon contact with the line in the check sheet. The “close-vicinity” refers to a situation in which the icons touch the line of the other icons and check sheets.

TABLE 7 is a template showing the meanings of the icons input to the computer. Based on TABLE 7, the presence or absence of an icon picked up and applied to the check sheet by the examinee is input. Incidentally, when the computer cannot determine the application state of the icons, it may be done by the person in charge according to TABLE 7.

The “designations” A-F in TABLE 7 have the following meanings.

A: Number designation

B: Selection and designation of specific icon

C: Arrangement designation

D: Protruding designation

E: Doll designation

F: Overlapping, Contact, Close-Vicinity designation

Based on TABLE 7, if there is any icon applied on the check sheet, its icon number is input. For example, “1” is input if there is “column” in the designation column B, and “0” is input if there is not. The “crowded arrangement”, “dispersive arrangement”, “balanced arrangement”, “deviative arrangement”, “bottom arrangement”, “upper arrangement”, “top-and-bottom arrangement” and “left-and-right arrangement” in the designation column C is determined based on the typical examples shown by reference in FIG. 18A to FIG. 18M, and “1” is input for any of these that is determined, and “0” is input for any that is not determined. If the computer is unable to make a determination, it may be done by the person in charge according to TABLE 7. Similarly, if the “protruding arrangement” is recognized in the designation column D, “1” is input, and “0” is input if it is not. In the designation column E, “1” is input if it is recognized as the “doll arrangement”, and “0” is input if it is not. If the “overlapping”, “contact” or “close-vicinity” is recognized in the designation column F, “1” is input, and “0” is input if it is not.

The definitions of the items in the columns C to F in TABLE 7 are as follows.

Crowded Arrangement: a case where many icons are gathered (in contact with each other or overlapped) in some areas (see the typical example in FIG. 18A).

Dispersive Arrangement: a case where icons are applied mostly distributed (see the typical example in FIG. 18B).

Balanced Arrangement: a case where they are applied one by one, for example, in each area in a balanced manner (see the typical example of FIG. 18C).

Deviative Arrangement: a case where the icons are applied in an overlapping manner so as to form a cluster (see the typical example in FIG. 18D).

Bottom Arrangement: a case where three or more icons are applied in the bottom area (including the bottom line) (see the typical example in FIG. 18E).

Upper Arrangement: a case where three or more icons are applied in the upper area (see the typical example in FIG. 18F).

Top-and-Bottom Arrangement: a case where icons are applied to the top and bottom areas, with no icons in the middle area (other than the top and bottom areas) (see the typical example in FIG. 18G).

Left-and-Right Arrangement: a case where icons are applied to the right and/or left side areas, with no icons in the middle area (other than the left and right side areas) (see the typical example in FIG. 18H).

Protruding Arrangement: a case where any icon extends beyond the line of the virtual frame, including a case where there is any icon stepping on the line of the virtual frame (see the typical example in FIG. 18I).

Doll Arrangement: a case where the face and circular icons are applied to specified positions (A4/A5), and another case where they are applied to form a doll (a head and a body) at other positions (see the typical example in FIG. 18J).

Overlapping: a case where icons are given in an overlapping manner (see the typical example in FIG. 18K).

Contact: a case where icons are in line-, face- or point-contact with each other (see the typical example in FIG. 18L).

Close-Vicinity: a case where three or more icons are applied in close vicinity, nearly in contact with each other (see the typical example in FIG. 18M).

FIGS. 18A to 18M show typical examples described in the “Sample” column in TABLE 7.

In the designation field C, normally one item is selected, but up to two items may still be selected. As a result, the arrangement state can be grasped first. However, with respect to the one representing the positions of the top, bottom, left and right, there is some combination which does not cause any inconsistency even in an overlapping manner, and therefore, the system is set up so as to allow such combination.

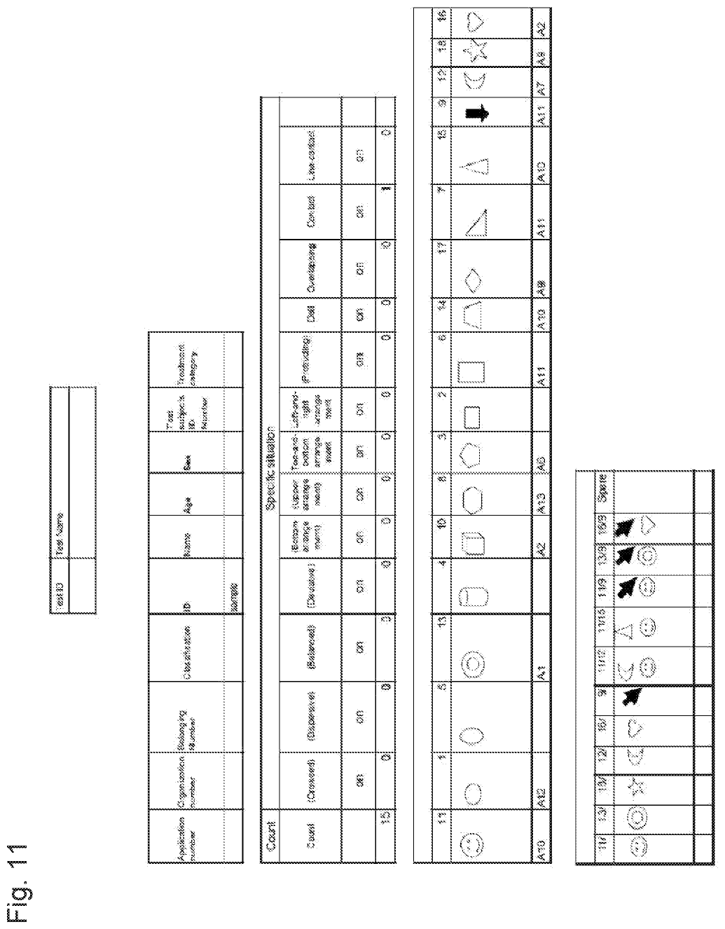

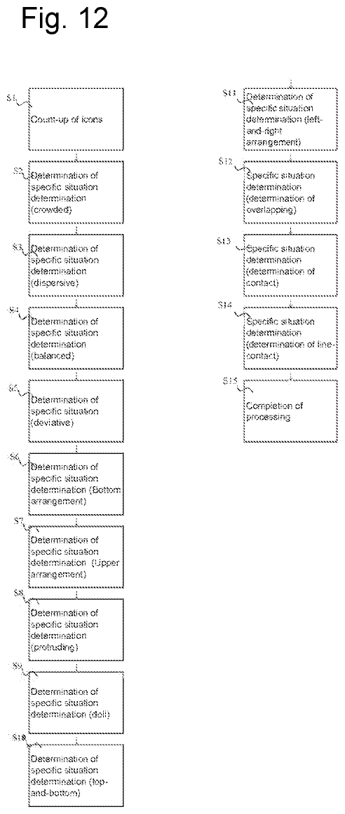

A computer 100 having the above-described configuration is used to determine an examinee's mental structure. An examinee applies a predetermined number (10-15) of desired icons to desired positions on the check sheet within a time limit (about 5 minutes). Based on the flow chart of FIG. 12, the check sheet to which the icons have been applied is processed by the computer in steps S1 to S15.

Next, a processing procedure by the computer 100 will be described with reference to FIG. 12. First, the total number of the icons applied to the check sheet is counted (“Count Up”·S1).

The specific situation of the icon application is then determined. The determination of the specific situation is a determination as to whether or not the icon application corresponds to all or part of the “Crowded arrangement” (S2), “Dispersive arrangement” (S3), “Balanced arrangement” (S4), “Deviative arrangement” (S5), “Bottom arrangement” (S6), “Upper arrangement” (S7), “Protruding arrangement” (S8), “Doll arrangement” (S9), “Top-and-Bottom arrangement” (S10), and “Left-and-Right arrangement” (S11). A series of determination steps of the “specific situation” (S2 to S11) is a step of extracting the application state of the icons.

If a “specific situation” is present (on), the determination is made as follows.

Crowded Arrangement: Strong dependence. Strong picky. Aggressive. Deflective. Envy jealousy. Density. Can't do something irresponsibly.

Dispersed Arrangement: scatter. Distraction of consciousness. Can't break out of my shell. Unknown anxiety. Can't depend on someone.

Balanced Arrangement: Equal Awareness, Averaging Awareness. Balance sensation, removing anxiety. Anxiety dispersion. Awareness of fragmentation.

Deviative Arrangement: Diffuse feeling. Doing as I like. Slack. Deviation of consciousness. indecision.

Bottom Arrangement: Stability orientation. Dependence. Seek peace of mind. Have confidence. Strong awareness of the current situation. No change.

Protruding Arrangement: Excessive consciousness, irresponsible. Hot flashes. Elimination of Subjects. Stray. confusing.

Upper Arrangement: Unstable consciousness. To be motivated. Hope. Desire. First . . . First-time . . . Virtual-Object.

Doll Arrangement: Human consciousness, targets. The person, this person, that person. Doll. Keepsake. Attack target

Left-and-Right Arrangement: unstable. Segregation awareness. Escape from the center. Fear of the center.

Top-and-Bottom Arrangement: Anxiety. Somehow frightened. Unknown Anxiety.

Next, the “overlapping” (S12), “contact” (S13) and “line-contact” (S14) arrangements are determined for the selected and applied icons. These steps determine the refractive directionality (“direction”) of the examinee's personality. For example, when the amount of the “overlapping” is large, it is determined that he/she tends to have a strong deflecting property, and when the amount of the “line-contact” is large, it is determined that he/she tends to be stable. The details are as follows.

Overlapping: Behavior under strong consciousness. Abnormal. Consciousness expression. Biased thought. Excessive consciousness. Ignore rules. Egocentric. Integration consciousness.

Contact: Feelings to approach of interest. Awareness of interval. Dependence. Look into empathy. Want a dialogue or relationship.

Line-contact: Strong dependence. Want to be stable. Escape from loneliness. Consciousness according to rules. Telepathy.

The determination of the doll arrangement is based on the sixth table (FIG. 10) in which the definitions for formations of face and body are stored. With respect to this determination, the person in charge manually modifies the processing result based on the above-mentioned criteria, if necessary.

Thus, when the processing for an examinee is completed (S15), the process returns to Step S1, and the above-described series of steps is performed for the next examinee.

Next, the content of evaluation in each step will be described. The count-up evaluation of the total icon number is as follows.

<1> If the count-up number is not limited, evaluation is performed as follows.

0: The response is evaluated as unresponsive or simply rejected (did not want to respond).

1˜17: It is evaluated as motivative, self-expressive and self-assertive (high).

18 (ALL): It is evaluated as self-assertive (fairly high), very motivative, very interested.

<2> There may be a limit on the number of count-up (10-18)

9 or less: It is evaluated as misunderstanding, not very responsive or simply rejected (did not want to respond).

10-17 (Selected): It is evaluated as motivated, self-expressive and self-assertive (much or high).

18 (ALL): It is evaluated as misunderstanding, self-assertive (fairly high), very motivated and very interested.

As described above, mental structure i.e. mental functions will include ones to be grasped as amygdala responses and others to be grasped as neocortex responses. A person responds to a stimulus by the amygdala earlier than by the neocortex by about 100 ms. The amygdala's responses include likes and dislikes, perceptions of dangers, aversions and strange feeling, etc. The amygdala emits dangerous signals in response to an alien substance (including unknown substances) entering the body, and reflex nerve motion or the like is immediately performed without passing through the neocortex.

On the other hand, the neocortex's response is recognized as being influenced by the experiences and knowledge of a certain individual. This means that what passed through the reaction of the amygdala is ultimately determined by the neocortex's response.

The present invention is based on the recognition that selection and arrangement of the icons include one resulting from the amygdala's response and another resulting from the neocortex's response, by which the analysis is performed.

In this regard, the overlapping, contact and line-contact is evaluated as follows in the specific situation interpretation steps.

“Overlapping” represents a condition in which a plurality of icons are overlapped, which may be regarded as a response by the amygdala. The selected and applied icons are recognized as representatives of people and/or goods, and the potential consciousness and tendency such as hiding and crushing are expressed by the overlapping.

“Contact” represents a condition in which the peripheral parts of the icons are in contact with each other, which may be regarded as a response by the neocortex. This can be identified as a response representing a close relationship but not reaching the overlapping.

“Line-contact” represents a condition in which the lines in the check sheet and the peripheral parts of the icons are in contact with each other, which may be regarded as a response by the amygdala. This is representative of strange feelings but is only occasional. This can be grasped as an unconscious reaction not much influenced by the neocortex.

“Close-vicinity” represents a condition in which an icon is in contact with another icon and/or the line components of the check sheet, which is the neocortex's response.

Considering that the amygdala's response occurs earlier than the neocortex's response by 100 ms, the specific situations of icon application are classified into the following three cases in different conditions, which will be evaluated as follows.

### <1> Icons Used and not Used

This reaction can be grasped as a reaction by an interest in the icons. Thus, the amygdala's response appears first (the sensory response precedes the neocortex's response). It also shows not only interest but also recognition of necessity, etc.

### <2> What is Affected by the Icons' Area and the Line Component of the Check Sheet

An examinee will apply icons while being influenced by the areas or the line components. That is, the presence or absence of an influence on the areas or the line components is an object of observation.

If the icon is applied not in an independent manner, not only the reaction by the amygdala but also the reaction by neocortex is added later. In other words, while cerebral reactions require trial and error, selection and application is an irreversible reaction, so as a result, it is observed that the amygdala's response occurs first.

### <3> Interaction Among Icons

Icons can be observed not only in an independent manner but also in an interactive manner such as “overlapping” and “contact”, and in some cases, icons may express the up-and-down or master-slave relationships.

The relationship between icons and line components of check sheets is the relationship between those that can be moved (icons) and those that cannot be moved (check sheets). In contrast, the icon-to-icon relationship differs only in that they can be moved, and because the amygdala's response precedes the neocortex's response by 100 ms, this will be affected by the resulting icon selections, as well as the response described in the preceding section. In other words, the interaction among icons is that the meaning of the existence of an icon alone disappears and the relationship between the icons is created. For example, star icons and moon icons are perceived to be located at the upper part because they are in a celestial body. The combination of a round icon and a square icon recalls a doll from its shape.

The crowded arrangement means that the icons concentrate on a particular area or at a point. The crowded arrangement includes different stages of close-crowding, contact-crowding and overlap-crowding, which represents introversion, including a demand to hide oneself in a group. This reaction is derived from likes and dislikes, which will first be managed by the amygdala.

The dispersive arrangement is a situation in which icons are not clustered and the distances between icons and line components remain constant. Dispersive arrangement expresses openness and extroversion. The neocortex is reached as if there were no amygdala responses, such as “freely, without restraint . . . ”. In other words, the dispersive arrangement is the response of neocortex.

The balanced arrangement is a case in which the icons are arranged in a well-balanced manner, such as one in each of the areas. This is a particular condition of the dispersive arrangement, which results from the neocortex's response. If neocortex has no sense of balance, the balanced arrangement cannot be formed.

Though not as much as the crowded arrangement, the deviative arrangement is a case in which the icons are concentrated slightly in a particular area. In other words, the deviative arrangement is a case in which the icon distribution is biased, which results preferentially from the amygdala's response. The neocortex has a little affect on the deviative arrangement, because it may be corrected later.

The bottom arrangement is a case in which the icons are located at the bottom of the virtual frame. The bottom arrangement is a stabilization-seeking reaction that is a response by the neocortex occurring later than the amygdala's response.

The upper arrangement is a case in which the icons are concentrated in the upper three areas. The upper arrangement retains a natural sense, and its relation to the icons is important. For example, when the star and moon icons are located in the upper areas, it will represent a normal sense because they are in sky. If the star or moon icons are located in the lower areas, it can be assessed that there is some effect from the neocortex. If many icons are placed on the upper areas, this represents anxiety. Anxiety is the neocortex's reaction, and it can be confirmed that this is much influenced by the later reaction.

The top-and-bottom arrangement is a case in which the icons are located in the upper and lower areas and there are few icons in the center. The top-and-bottom arrangement emerges as a result of being influenced by the likes and dislikes and a strange feeling. This will mainly result from the amygdala's response.

The left-and-right arrangement is a case in which the icons are located in the left and right areas, which is a logical response by the neocortex. The top-and-bottom arrangement and the left-and-right arrangement would be found in a limited number of cases.

The protruding arrangement is classified into complete out-of-virtual-frame protrusion and partial protrusion. The complete protrusion is a reaction preferentially by the amygdala, which may be understood that something in the subconscious becomes explicit and then is reacted to. The partial protrusion is a reaction preferentially by the neocortex rather than by the amygdala, including hesitation.

The doll arrangement is a case in which the icon located in the lower area is square and the icon located in the upper area is circular. The doll arrangement is a reaction by the neocortex, in which something originally in the subconscious becomes explicit.

Since every icon has its own meaning as shown in TABLE 3A and TABLE 3B, selecting an icon is interpreted as being introspective because it reflects mental function and state. On the other hand, it is understood as being reflective, if the icons are deemed to be arranged in some interactive manner with a specific position, angle and the like in the check sheet that is an environment. Similarly, the interaction among icons can be grasped as being reflective. In other words, “introspective” means a looking-back by oneself on the function and state of one's own mind, and “reflective” means that meanings and interpretations are given from outside without looking back by oneself on the function and state of one's own mind.

Because the amygdala's responses occur earlier than the neocortex's responses by 100 ms, the sensory system reacts earlier than thinking, analyzing and judging by the neocortex. In other words, the retina reacts thorough visual information, which is transmitted to the amygdala. Then, also thanks to the neocortex's response, the brain expresses a potential response. Icon application also involves a bored response with time, but if interested or motivated, the response persists and the icon application increases. If there is a limit on the count-up number of the icons, the neocortex will respond in a similar manner within the limits.

FIG. 11 shows the content of the process specification table stored in the memory 104, and the processed result is output as a test result as follows.

Icon part (count: the icon number applied)

Icon part (specific situation)

Icon part (positional state of icon application)

Icon part (state of adopted icons alone and in combination)

The computer-processed data is stored in the headquarters computer 100 as a test result according to the present invention.

The order of the icons shown in FIG. 11 will be described.

In FIG. 11, the icons are shown in the order of from the rounded icons to the angular icons from left to right. Icon No. 12 (moon), Icon No. 18 (star), and Icon No. 16 (heart shape) are shown in this order from left to right.

### Examples

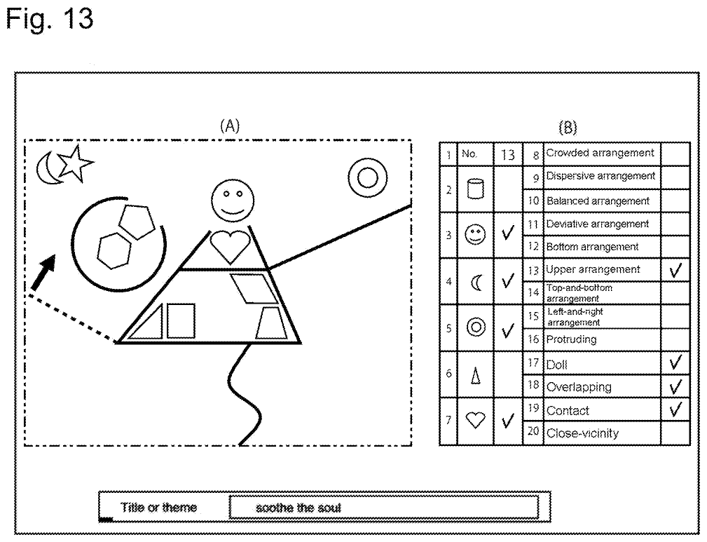

FIGS. 13 to 17 show different examinees' examples. In each of the drawings, part (A) shows an example in which an icon is applied by the examinee, part (B) shows processing of the in-computer data obtained by totaling the locations of the applied icons.

In FIG. 13, part (A), the application state of the icons by the examinee 0001 will be recognized as follows based on TABLE 7.

TABLE 9

| A (count-up): 13 | | |
|---|---|---|
| B (select specific icon) and its | Location of | Definitions of |
| occurrences: | specific | FIG. 3A to FIG. 3L |
| Selected designation of specific | situation | |
| icon: | appearance: | |
| Icon No. 11 (Face): | Area A-5: | Doll |
| Icon No. 12 (Moon): | Area A-7: | Released |
| Icon No. 13 (doughnut): | Area A-9: | Released |
| Icon No. 16 (heart shape): | Area A-4: | Manifestation of |
| | | intentional objects |
| C-F (Arrangement designation, etc.): | | |
| Crowded arrangement: 0 | | |
| Dispersive arrangement: 0 | | |
| Balanced arrangement: 0 | | |
| Deviative arrangement: 0 | | |
| Bottom arrangement: 0 | | |
| Upper arrangement: 1 | | |
| Top-and-bottom arrangement: 0 | | |
| Left-and-right arrangement: 0 | | |
| Protruding arrangement: 0 | | |
| Doll arrangement: 1 | | |
| Overlapping: 1 | | |
| Contact: 1 | | |
| Line-contact: 0 | | |

Therefore, at this stage, the examinee 0001 is determined as follows.

TABLE 10

| | Appearance | |
|---|---|---|
| | of specific | |
| Selected designation of | situation | |
| specific icon: | Location | |
| (0001) | (Area) | Definitions |
| Count: 13 | A5 | Moderate motivation |
| Icon No. 11 (Face) | | Not rejecting human |
| | | relationships |
| Icon No. 13 (doughnut) | A9 | Strange feeling, |
| | | weakness, etc. |
| Icon No. 12 (Moon) | A7 | Be able to make |
| | | common sense |
| | | decisions and |
| | | thinkinging |
| Icon No. 16 (heart shape) | A4 | Be kind and be able |
| | | to consider |
| Upper arrangement | | Be somewhat worried |
| | | about the situation |
| Doll arrangement | | High capacity for |
| | | storage, analysis, |
| | | judgment, etc. |
| Overlapping | | Be able to maintain |
| | | a degree of human |
| | | relationships |
| Contact | | Be able to interact |
| | | with others at |
| | | reasonable distances |

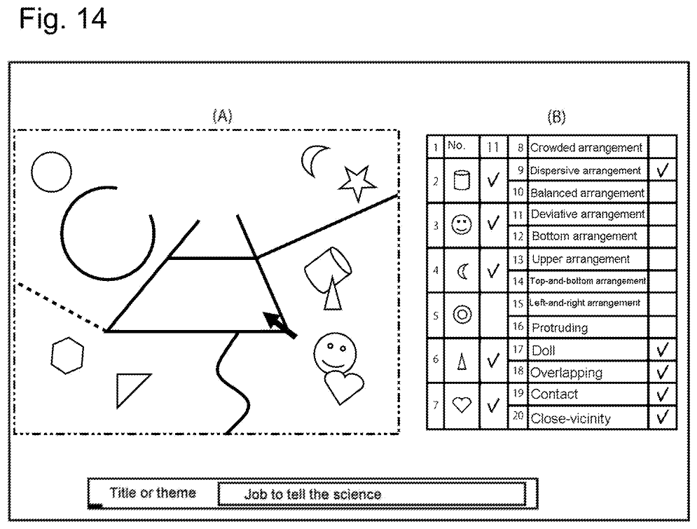

In FIG. 14, part (A), the application state of the icons by the examinee 0002 will be recognized as follows based on TABLE 7.

TABLE 11

| A (count-up): 11 | | |
|---|---|---|
| B (select specific icon) and its | Location of | Definitions of |
| occurrences: | specific | FIG. 3A to FIG. 3L |
| Selected designation of specific | situation | |
| icon: | appearance: | |
| Icon No. 4 (column): | Area A-10: | afford, no problem |
| Icon No. 11 (Face): | Area A-11: | Certain people |
| | | are irrelevant |
| Icon No. 12 (Moon): | Area A-9: | Released |
| Icon No. 15 (isosceles triangle): | Area A-10: | Afford, no problem |
| Icon No. 16 (heart shape): | Area A-11: | Feeling that no |
| | | one will understand |
| Icon No. 9 (arrow): | Contact | I realize that it |
| | point X-5 | is somehow bad for |
| | (intersection | me. |
| | of line L9 | |
| | and TAL17): | |
| C-F (Arrangement designation, etc.): | | |
| Crowded arrangement: 0 | | |
| Dispersive arrangement: 1 | | |
| Balanced arrangement: 0 | | |
| Deviative arrangement: 0 | | |
| Bottom arrangement: 0 | | |
| Upper arrangement: 0 | | |
| Top-and-bottom arrangement: 0 | | |
| Left-and-right arrangement: 0 | | |
| Protruding arrangement: 0 | | |
| Doll arrangement: 1 | | |
| Overlapping: 1 | | |
| Contact: 1 | | |
| Line-contact: 1 | | |

Thus, the examinee 0002 is determined at this stage as follows.

TABLE 12

| | Appearance | |
|---|---|---|
| | of specific | |
| Selected designation of | situation | |
| specific icon: | Location | |
| (0002) | (Area) | Definitions |
| Count: 11 | | Somewhat lower desire |
| Icon No. 11 (Face) | A-10/11 | Not rejecting human |
| | | relationships |
| Icon No. 12 (Moon) | A9 | Be able to make common |
| | | sense decisions and |
| | | thinkinging |
| Icon No. 16 (heart shape) | A11 | Be kind and be able to |
| | | consider |
| Icon No. 4 (column) | A10 | It originally represents a |
| | | sense of presence, but here |
| | | is a reflecting telescope |
| Icon No. 15 | A10 | Used as a balance stand |
| (isosceles triangle) | | (of a reflecting telescope) |
| Icon No. 4 (column) + | — | Icon No. 4 (column) and icon |
| Icon No. 15 | — | Combinations with No. 15 |
| (isosceles triangle) | | (isosceles triangle) |
| | | represent something like |
| | | reflective telescopes |
| | | (not have deep meaning) |
| Dispersive arrangement | | Open, not persistent |
| Doll arrangement | | High capacity for |
| | | storage, analysis, |
| | | judgment, etc. |
| Overlapping | | Be able to maintain a |
| | | degree of human |
| | | relationships |
| Contact | | Be able to interact |
| | | with others at |
| | | reasonable distances |
| Line-contact | | I have a dependence. |

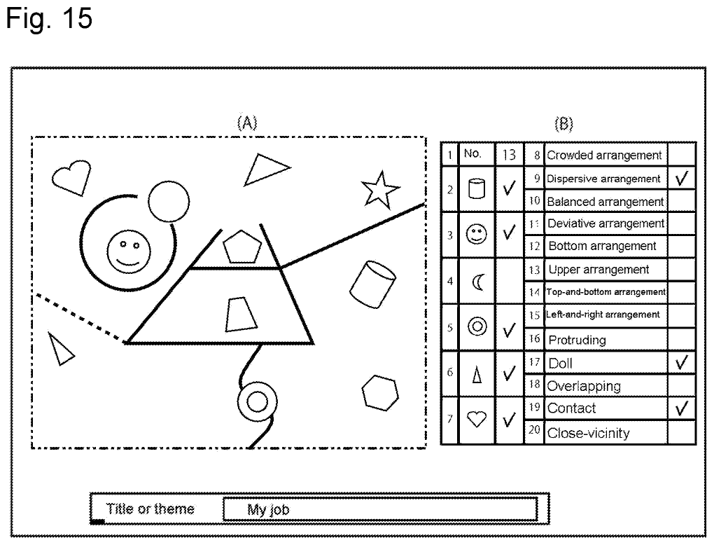

In FIG. 15, part (A), the application state of the icons by the examinee 0003 will be recognized as follows based on TABLE 7.

TABLE 13

| A (count-up): 13 | | |
|---|---|---|
| B (select specific icon) and its | Location of | Definitions of |
| occurrences: | specific | FIG. 3A to FIG. 3L |
| Selected designation of specific | situation | |
| icon: | appearance: | |
| Icon No. 4 (column): | Area A-10: | Afford, no problem |
| Icon No. 11 (Face): | Area A-1: | Certain people |
| | | are irrelevant. |
| Icon No. 13 (doughnut): | Lines L-16: | |
| Icon No. 15 (isosceles triangle): | Area A-13: | The sense of |
| | | destruction and |
| | | aggression do not |
| | | vanish. |
| Icon No. 16 (heart shape): | Area A-7: | Released |
| C-F (Arrangement designation, etc.): | | |
| Crowded arrangement: 0 | | |
| Dispersive arrangement: 1 | | |
| Balanced arrangement: 0 | | |
| Deviative arrangement: 0 | | |
| Bottom arrangement: 0 | | |
| Upper arrangement: 0 | | |
| Top-and-bottom arrangement: 0 | | |
| Left-and-right arrangement: 0 | | |
| Protruding arrangement: 0 | | |
| Doll arrangement: 1 | | |
| Overlapping: 0 | | |
| Contact: 1 | | |

Thus, the examinee 0003 is determined at this stage as follows.

TABLE 14

| | Appearance | |
|---|---|---|
| | of specific | |
| Selected designation of | situation | |
| specific icon: | Location | |
| (0003) | (Area) | Definitions |
| Count:13 | | Moderate motivation |
| Icon No. 11 (Face) | A-1 | Not rejecting human |
| | | relationships |
| Icon No. 13 (doughnut) | A-11/12 | Strange feeling, |
| | | weakness, etc. |
| Icon No. 4 (column) | A10 | It is interpreted |
| | | as make somebody do |
| Icon No. 15 | A13 | The inclination itself |
| (isosceles triangle) | | is interpreted as |
| | | representing the test |
| | | subject's instability. |
| Icon No. 16 (heart shape) | A7 | Be kind and be able |
| | | to consider |
| Dispersive arrangement | | Open, not persistent |
| Doll arrangement | | High capacity for |
| | | storage, analysis, |
| | | judgment, etc. |
| Contact | | Interact with others |
| | | at reasonable distances |

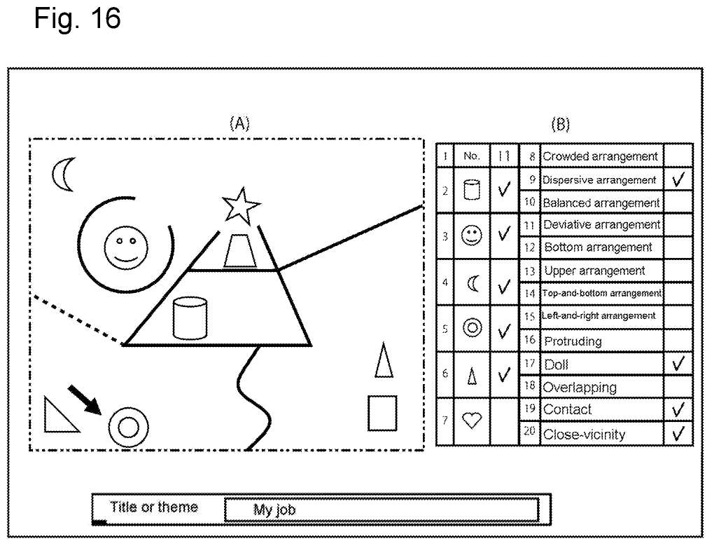

In FIG. 16, part (A), the application status of the icons by the examinee 0004 will be recognized as follows based on TABLE 7.

TABLE 15

| A (count-up): 11 | | |
|---|---|---|
| B (select specific icon) and its | Location of | Definitions of |
| occurrences: | specific | FIG. 3A to FIG. 3L |
| Selected designation of specific | situation | |
| icon: | appearance: | |
| Icon No. 4 (column): | Area A-3: | I want to target |
| | | something |
| Icon No. 11 (Face): | Area A-1: | I want to |
| | | monopolize or |
| | | target a |
| | | particular person. |
| Icon No. 12 (Moon): | Area A-7: | Released |
| Icon No. 13 (doughnut): | Area A-13: | Wheels, cars |
| Icon No. 15 (isosceles triangle): | Area A-11: | The sense of |
| | | destruction and |
| | | aggression do not |
| | | vanish. |
| Icon No. 9 (arrow): | Area A-13: | Be aware |
| C-F (Arrangement designation, etc.): | | |
| Crowded arrangement: 0 | | |
| Dispersive arrangement: 1 | | |
| Balanced arrangement: 0 | | |
| Deviative arrangement: 0 | | |
| Bottom arrangement: 0 | | |
| Upper arrangement: 0 | | |
| Top-and-bottom arrangement: 0 | | |
| Left-and-right arrangement: 0 | | |
| Protruding arrangement: 0 | | |
| Doll arrangement: 1 | | |
| Overlapping: 0 | | |
| Contact: 1 | | |
| Line-contact: 1 | | |

Thus, the examinee 0004 is determined at this stage as follows.

TABLE 16

| | Appearance | |
|---|---|---|
| | of specific | |
| Selected designation of | situation | |
| specific icon: | Location | |
| (0004) | (Area) | Definitions |
| Count:11 | | Distraction, excessive |
| | | consciousness |
| Icon No. 11 (Face) | A-1 | Not rejecting human |
| | | relationships |
| Icon No. 13 (doughnut) | A-12 | Strange feeling, |
| | | weakness, etc. |
| Icon No. 4 (column) | A-3 | The presence without |
| | | inclination in closed |
| | | space represents a |
| | | sense of loneliness |
| Icon No. 12 (Moon) | A-7 | Be able to make common |
| | | sense decisions and |
| | | thinking |
| Icon No. 15 (isosceles | A-10 | Have image of a sharp, |
| triangle) | | dangerous, or sticking, |
| | | etc. |
| Icon No. 6 (rectangle) | A-11 | It indicates unusual |
| | | things. |
| | | Differences from |
| | | others, etc. |
| Icon No. 15 (isosceles | — | Associate a small house |
| triangle) + Icon No. 6 | | |
| Dispersive arrangement | | Open, not persistent |
| Doll arrangement | | High capacity for |
| | | storage, analysis, |
| | | judgment, etc. |
| Contact | | Interact with others at |
| | | reasonable distances |
| Line-contact | | I have a dependence. |

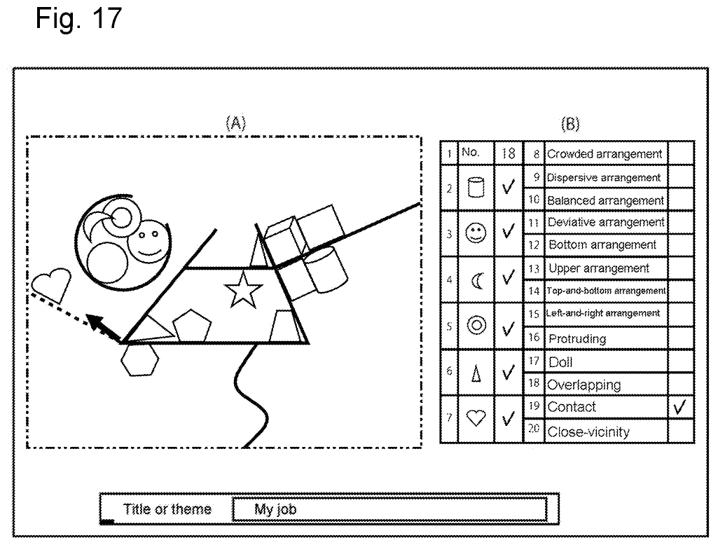

In FIG. 17, part (A), the application state of the icons by the examinee 0005 will be recognized as follows based on TABLE 7.

TABLE 17

| A (count-up): 18 | | |
|---|---|---|
| B (select specific icon) and its | Location of | Definitions of |
| occurrences: | specific | FIG. 3A to FIG. 3L |
| Selected designation of specific | situation | |
| icon: | appearance: | |
| Icon No. 4 (column): | Area A-10: | Afford, no problem |
| Icon No. 11 (Face): | Area A-1: | I want to |
| | | monopolize or target |
| | | a particular person. |
| Icon No. 12 (Moon): | Area A-1: | Coexistence with |
| | | anxiety |
| Icon No. 13 (doughnut): | Area A-1: | I want to target |
| | | something |
| Icon No. 15 (isosceles triangle): | Area A-4: | Dangerous elements |
| | | that cannot be |
| | | controlled |
| Icon No. 16 (heart shape): | Area A-6: | Tight. |
| Icon No. 4 (column) | Line A-10: | Afford, no problem |
| C-F (Arrangement designation, etc.): | | |
| Crowded arrangement: 0 | | |
| Dispersive arrangement: 0 | | |
| Balanced arrangement: 0 | | |
| Deviative arrangement: 0 | | |
| Bottom arrangement: 0 | | |
| Upper arrangement: 0 | | |
| Top-and-bottom arrangement: 0 | | |
| Left-and-right arrangement: 0 | | |
| Protruding arrangement: 0 | | |
| Doll arrangement: 0 | | |
| Overlapping: 0 | | |
| Contact: 1 | | |
| Line-contact: 0 | | |

Thus, the examinee 0005 is determined at this stage as follows.

TABLE 18

| | Appearance | |
|---|---|---|
| | of specific | |
| Selected designation of | situation | |
| specific icon: | Location | |
| (0005) | (Area) | Definitions |
| Count:18 | | Distraction, |
| | | excessive |
| | | consciousness |
| Icon No. 11 (Face) | A-1 | Not rejecting |
| | | human relationships |
| Icon No. 13 (doughnut) | A-1 | Strange feeling, |
| | | weakness, etc. |
| Icon No. 4 (column) | A-10 | Be used as representing |
| | | a strong dependence |
| Icon No. 15 (isosceles | A-4 | It represents a |
| triangle) | | persistent dependence |
| | | similarly |
| Icon No. 12 (Moon) | A-1 | Be able to make |
| | | common sense decisions |
| | | and thinking |
| Icon No. 16 (heart shape) | | Be kind and be able |
| | | to consider |
| There is a large contact | A-6 | It indicates a |
| of icons. | | strong dependence and |
| | | a strong dependability |

Each of the above-described data shown in FIGS. 13 to 17 is stored in the headquarters computer 100 as the inspection result, which can be used as a material for further investigation.

The standard test time for icon application according to the present invention is about 5 minutes. In addition, there are restrictions on the age of examinees, and those who are between working age and 60 years old are eligible for inspection, especially those who are up to about 45 years old.

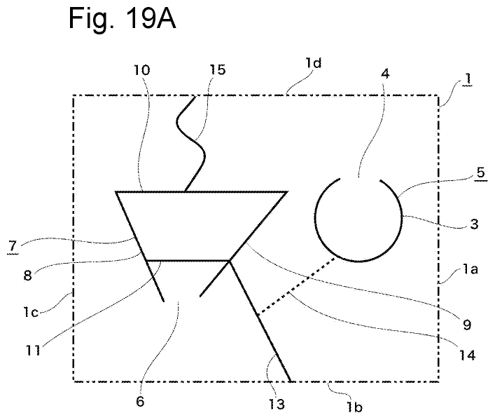

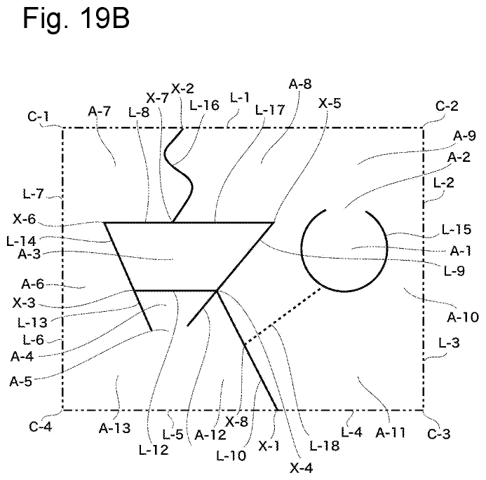

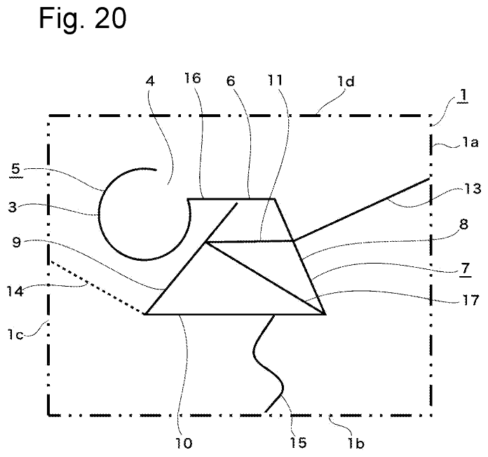

The present invention is not limited to the above-described embodiments. For example, the shape of the figure in which the areas are partitioned is not limited to that shown in FIGS. 1A and 1B and may include various embodiments such as shown in FIGS. 19A and 19B and FIG. 20, for example. In addition, in the embodiments described above, the examinee applies icons to a check sheet electronically via terminal 200, and the result is transmitted to computer 100. Alternatively, for example, the check sheet can be on paper, and the icons can be stickers that are physically applied to the check sheet. The resulting check sheet can be scanned for input to the computer 100, or the application of the icons to the check sheet can be input in other ways to the computer 100.

In FIG. 19A, the boundary elements depicted in a rectangular window 1 consisting of window virtual lines 1a to 1d comprise a circular FIG. 5 having a cut-off 4 at the upper part of a curved circumferential line 3, a triangular FIG. 7 having a cut-off 6 at the apex thereof and an intermediate straight line 11 extending in parallel with a straight bottom line 10 between opposite oblique-side lines 8, 9, a straight line 13 (inclined straight line) extending obliquely down and to right from the right end of the intermediate line 11 to the window virtual line 1b, a broken line 14 (inclined broken line) extending obliquely up and to right from the middle portion of the inclined straight line 13 to the lower part of the circular FIG. 5, and a S-shape curved line 15 extending up from the middle portion of the bottom 10 to the window virtual line 1d. With these boundary elements, the above-mentioned areas are divided into nine sectional areas, among which three are located almost in a horizontal direction and other three almost in a vertical direction. The cut-off 4 opens upward and the cut-off 6 opens downward.

In FIG. 20, the boundary elements depicted in a rectangular window 1 consisting of window virtual lines 1a to 1d comprises a circular FIG. 5 having a cut-off 4 at the upper-right part of a curved circumferential line 3, a triangular FIG. 7 having a cut-off 6 at the apex thereof and an intermediate straight line 11 extending in parallel with a straight bottom line 10 between opposite oblique-side lines 8, 9, a straight line 13 (inclined straight line) extending obliquely up and to right from the right end of the intermediate line 11 to the window virtual line 1a, a broken line 14 (inclined broken line) extending obliquely up and to left from the left end (opposite to the inclined straight line 13) of the bottom 10 to the window virtual line 1c, a S-shape curved line 15 extending down from the rightward middle portion of the bottom 10 to the window virtual line 1b, another straight line 16 (connecting line) connecting one end of the cut-out 4 of the circular FIG. 5 to one end of the cut-out 6 of the triangular FIG. 7, and another straight line 17 (inclined line) extending obliquely between the left end (opposite to the inclined straight line 13) of the intermediate line 11 and the right end of the bottom 10. With these boundary elements, the areas are divided into nine sectional areas, among which three are located almost in a horizontal direction and other three almost in a vertical direction. Both cut-offs 4 and 6 open upward.

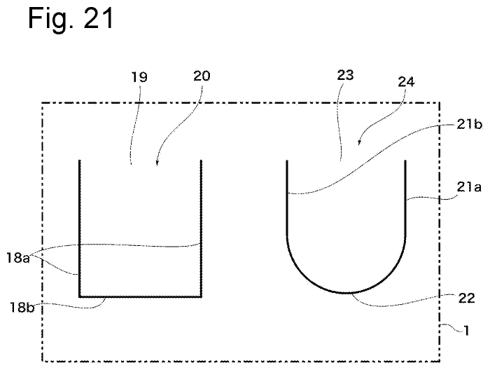

FIG. 21 shows another embodiment wherein only a part of the above-mentioned boundary elements is used. In this embodiment, the boundary elements depicted in the window 1 comprise a quadrangular hole 20 at the left having straight lines 18a and a straight line 18b perpendicular thereto with an opening at the top 19, and a U-shaped hole 24 at the right having parallel straight lines 21a, 21b and a semi-circular curved line 22 connecting the lower ends of the straight lines 21a, 21b with an opening at the top 23.

In the case of the check sheet as shown in FIG. 21, a relatively simple investigation is performed, which makes it possible to reduce the burden on the examinee. On the other hand, when a check sheet in which a lot of the boundary elements are depicted to form a lot of areas as shown in FIGS. 1A and 1B, 19A and 19B, and 20, the investigation becomes relatively complicated, which makes it possible to perform a wide range of investigations and therefore to obtain various analytical results.

The visual approach-based aptitude testing system according to the present invention can be used, for example, for judgement of vocational aptitude of personnel in entrance examinations (including mid-career recruitment examinations) and promotion examinations in companies and government offices. It may also be used for personnel evaluation.

1. A visual approach-based aptitude testing system comprising a computer connected to a terminal of an examinee through a communication network,
wherein the computer includes:
a memory which stores (i) information for creating a display screen including a check sheet in which a plurality of areas are formed by boundary elements depicted in a frameless test area and which comprises graphic figures having no inherent meaning, and a plurality of icons applicable to the check sheet, and (ii) a first table including definitions of the boundary elements in the check sheet, a second table including kinds, a number, and definitions of the icons, and a third table including definitions of meanings of positions of the icons in the check sheet; and
a processor which executes a program to perform operations including:
providing the information for creating the display screen to the examinee's terminal, so that the examinee's terminal displays the display screen including the check sheet and the plurality of icons;
sequentially writing, to the memory, test data including icons applied by the examinee to the check sheet via the terminal, and an examinee's ID transmitted from the terminal;
sequentially reading out the test data and the examinee's ID stored in the memory to count a total number of the icons applied to the check sheet;
executing discrimination of whether or not an arrangement of the icons on the check sheet in the read-out test data falls under the definitions in the tables stored in the memory; and
storing results of the discrimination in the memory to be used for evaluation of the examinee's aptitude based on the total number of the icons applied to the check sheet and the arrangement of the icons;
wherein the boundary elements, which are depicted in the frameless test area and form the plurality of areas, comprise (i) a circular figure having a cut-out at a part of its circumference, (ii) a triangular figure having a cut-out at its apex and an intermediate line extending between opposite oblique side lines of the triangular figure and in parallel with a bottom of the triangular figure, (iii) a straight line extending obliquely upward and rightward from one end of the intermediate line to a window virtual line, (iv) a broken line extending obliquely upward and leftward from one end of the bottom of the triangular figure to a window virtual line, and (v) an S-shaped curved line extending downward from the bottom of the triangular figure to a window virtual line.

2. The visual approach-based aptitude testing system according to claim 1, wherein the memory further stores a fourth table including meanings of positional relationships between the icons or between the icons and lines in the check sheet, and
wherein the operations performed by the processor further include executing discrimination of whether or not the positional relationships exist in the arrangement of the icons in the read-out test data, with reference to the fourth table.

3. The visual approach-based aptitude testing system according to claim 2, wherein the positional relationships comprise at least one of an overlapping relationship, a contact relationship, a line-contact relationship, and a close-vicinity relationship.

4. The visual approach-based aptitude testing system according to claim 1, wherein the memory further stores a fifth table including meanings of the applied icons forming specific patterns, and
wherein the operations performed by the processor further include executing discrimination of whether or not one of the specific patterns exists in the icons applied to the check sheet.

5. The visual approach-based aptitude testing system according to claim 4, wherein the specific patterns include at least one of a crowded arrangement, a dispersive arrangement, a balanced arrangement, a deviative arrangement, a bottom arrangement, an upper arrangement, a top-and-bottom arrangement, a left-and-right arrangement, a protruding arrangement, and a doll arrangement.

6. The visual approach-based aptitude testing system according to claim 1, wherein the memory further stores a sixth table including a meaning of an appearance of a specific one of the icons, and
wherein the operations performed by the processor further include executing discrimination whether or not the specific icon appears.

7. A visual approach-based aptitude testing system comprising a computer connected to a terminal of an examinee through a communication network,
wherein the computer includes:
a memory which stores (i) information for creating a display screen including a check sheet in which a plurality of areas are formed by boundary elements depicted in a frameless test area and which comprises graphic figures having no inherent meaning, and a plurality of icons applicable to the check sheet, and (ii) a first table including definitions of the boundary elements in the check sheet, a second table including kinds, a number, and definitions of the icons, and a third table including definitions of meanings of positions of the icons in the check sheet; and
a processor which executes a program to perform operations including:
providing the information for creating the display screen to the examinee's terminal, so that the examinee's terminal displays the display screen including the check sheet and the plurality of icons;
sequentially writing, to the memory, test data including icons applied by the examinee to the check sheet via the terminal, and an examinee's ID transmitted from the terminal;
sequentially reading out the test data and the examinee's ID stored in the memory to count a total number of the icons applied to the check sheet;
executing discrimination of whether or not an arrangement of the icons on the check sheet in the read-out test data falls under the definitions in the tables stored in the memory; and
storing results of the discrimination in the memory to be used for evaluation of the examinee's aptitude based on the total number of the icons applied to the check sheet and the arrangement of the icons;
wherein the boundary elements, which are depicted in the frameless test area and form the plurality of areas, comprise (i) a circular figure having a cut-out at a part of its circumference, (ii) a triangular figure having a cut-out at its apex and an intermediate line extending between opposite oblique side lines of the triangular figure and in parallel with a bottom of the triangular figure, (iii) a straight line extending obliquely downward from one end of the intermediate line to a window virtual line, (iv) a broken line extending obliquely upward and rightward from an intermediate location on the straight line to a lower portion of the circular figure, and (v) an S-shaped curved line extending upward from the bottom of the triangular figure to a window virtual line.

8. The visual approach-based aptitude testing system according to claim 7, wherein the memory further stores a fourth table including meanings of positional relationships between the icons or between the icons and lines in the check sheet, and
wherein the operations performed by the processor further include executing discrimination of whether or not the positional relationships exist in the arrangement of the icons in the read-out test data, with reference to the fourth table.

9. The visual approach-based aptitude testing system according to claim 8, wherein the positional relationships comprise at least one of an overlapping relationship, a contact relationship, a line-contact relationship, and a close-vicinity relationship.

10. The visual approach-based aptitude testing system according to claim 7, wherein the memory further stores a fifth table including meanings of the applied icons forming specific patterns, and
wherein the operations performed by the processor further include executing discrimination of whether or not one of the specific patterns exists in the icons applied to the check sheet.

11. The visual approach-based aptitude testing system according to claim 10, wherein the specific patterns include at least one of a crowded arrangement, a dispersive arrangement, a balanced arrangement, a deviative arrangement, a bottom arrangement, an upper arrangement, a top-and-bottom arrangement, a left-and-right arrangement, a protruding arrangement, and a doll arrangement.

12. The visual approach-based aptitude testing system according to claim 7, wherein the memory further stores a sixth table including a meaning of an appearance of a specific one of the icons, and
wherein the operations performed by the processor further include executing discrimination whether or not the specific icon appears.

13. A visual approach-based aptitude testing system comprising a computer connected to a terminal of an examinee through a communication network,
wherein the computer includes:
a memory which stores (i) information for creating a display screen including a check sheet in which a plurality of areas are formed by boundary elements depicted in a frameless test area and which comprises graphic figures having no inherent meaning, and a plurality of icons applicable to the check sheet, and (ii) a first table including definitions of the boundary elements in the check sheet, a second table including kinds, a number, and definitions of the icons, and a third table including definitions of meanings of positions of the icons in the check sheet; and
a processor which executes a program to perform operations including:
providing the information for creating the display screen to the examinee's terminal, so that the examinee's terminal displays the display screen including the check sheet and the plurality of icons;
sequentially writing, to the memory, test data including icons applied by the examinee to the check sheet via the terminal, and an examinee's ID transmitted from the terminal;
sequentially reading out the test data and the examinee's ID stored in the memory to count a total number of the icons applied to the check sheet;
executing discrimination of whether or not an arrangement of the icons on the check sheet in the read-out test data falls under the definitions in the tables stored in the memory; and
storing results of the discrimination in the memory to be used for evaluation of the examinee's aptitude based on the total number of the icons applied to the check sheet and the arrangement of the icons;
wherein the boundary elements, which are depicted in the frameless test area and form the plurality of areas, comprise (i) a circular figure having a cut-out at a part of its circumference, (ii) a triangular figure having a cut-out at its apex and an intermediate line extending between opposite oblique side lines of the triangular figure and in parallel with a bottom of the triangular figure, (iii) a first straight line extending obliquely upward from a first end of the intermediate line to a window virtual line, (iv) a broken line extending obliquely upward and leftward from a first end of the bottom of the triangular figure to a window virtual line, (v) an S-shaped curved line extending downward from an intermediate portion of the bottom of the triangular figure to a window virtual line, (vi) a second straight line extending from an end of the cut-out of the circular figure to an end of the cutout of the triangular figure, and (vii) a third straight line which is inclined and extends from a second end of the intermediate line to a second end of the bottom of the triangular figure.

14. The visual approach-based aptitude testing system according to claim 13, wherein the memory further stores a fourth table including meanings of positional relationships between the icons or between the icons and lines in the check sheet, and
wherein the operations performed by the processor further include executing discrimination of whether or not the positional relationships exist in the arrangement of the icons in the read-out test data, with reference to the fourth table.

15. The visual approach-based aptitude testing system according to claim 14, wherein the positional relationships comprise at least one of an overlapping relationship, a contact relationship, a line-contact relationship, and a close-vicinity relationship.

16. The visual approach-based aptitude testing system according to claim 13, wherein the memory further stores a fifth table including meanings of the applied icons forming specific patterns, and
wherein the operations performed by the processor further include executing discrimination of whether or not one of the specific patterns exists in the icons applied to the check sheet.

17. The visual approach-based aptitude testing system according to claim 16, wherein the specific patterns include at least one of a crowded arrangement, a dispersive arrangement, a balanced arrangement, a deviative arrangement, a bottom arrangement, an upper arrangement, a top-and-bottom arrangement, a left-and-right arrangement, a protruding arrangement, and a doll arrangement.

18. The visual approach-based aptitude testing system according to claim 13, wherein the memory further stores a sixth table including a meaning of an appearance of a specific one of the icons, and
wherein the operations performed by the processor further include executing discrimination whether or not the specific icon appears.
